(19) United States
(12) Patent Application Publication
(10) Pub. No.: US 2024/0287622 A1
(43) Pub. Date: Aug. 29, 2024

(54) METHOD FOR BROAD ANALYSIS OF MICROORGANISMS AND DRUG-RESISTANCE DETERMINANTS IN SAMPLES FROM A SUBJECT AND DEVICES THEREFOR

(71) Applicant: Janus-I Science Inc., Vista, CA (US)

(72) Inventors: Thomas A. Hall, Vista, CA (US); Lisa M. Risen, Vista, CA (US); Michael R. Mosel, Vista, CA (US); Christine V. Marzan, Vista, CA (US); James C. Hannis, Vista, CA (US); David J. Ecker, Vista, CA (US)

(21) Appl. No.: 18/573,317

(22) PCT Filed: Jun. 22, 2022
(86) PCT No.: PCT/US2022/034573

§ 371 Date: Dec. 21, 2023

Related U.S. Application Data
(60) Provisional application No. 63/213,640, filed on Jun. 22, 2021.

(51) Int. Cl. C12Q 1/689; C12N 15/10

(52) U.S. Cl. CPC C12Q 1/689; C12N 15/1006

(57) ABSTRACT

Disclosed herein are methods for detecting the presence, identifying, and/or quantifying amount of an unidentified microbial agent in a sample from a subject.

This application claims the benefit of U.S. Provisional Application Ser. No. 63/213,640 filed Jun. 21, 2021, the entirety of which is hereby incorporated by reference herein.

All publications, patents, and patent applications mentioned in this specification are herein incorporated by reference to the same extent as if each individual publication, patent, of patent application was specifically and individually indicated to be incorporated by reference.

### BACKGROUND OF THE INVENTION

Blood culture followed by biochemical and/or molecular analysis for pathogen identification are known. However, there remains a need for agnostic identification of blood-borne pathogens and antimicrobial resistance determinants through universal and broad amplification targeting of bacteria and fungi with a calibrated quantification of pathogen load directly tied to the original source sample, as opposed to only quantification of nucleic acid input to a molecular reaction. This invention addresses that need.

### SUMMARY

The invention provides methods and devices for an end-to-end process for analyzing liquid samples (such as blood or lung aspirates) for the presence of virtually any bacterial or fungal sepsis-associated agent (e.g., see FIG. 1), with quantification of pathogen load within the source sample. The back-end process is designed to also be compatible with any biological sample containing microorganisms and can be adapted to wound or swab specimens as well as expanded to include viral pathogens and/or parasites. The invention includes the modular incorporation of analysis of antimicrobial resistance (AMR) factors.

### BRIEF DESCRIPTION OF FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 Schematic overview of JIS Sepsis analysis workflow.

FIG. 2 JIS cell lysis device.

FIG. 3 Example of partial HTML, report generated by NanoSepsID.

FIG. 4 NanoSepsID report for a Klebsiella pneumoniae isolate carrying a KPC carbapenamase and extracted from human Blood.

FIG. 5 NanoSepsID) report for a negative control development test demonstrating identification and source quantification of E. coli background contamination in synthetic calibrant constructs prepared as plasmids in E. coli.

FIG. 6. NanoSepsID report for contrived samples spiked into hetastarch-cleared human blood of the ESKAPE organisms A. Enterococcus faecium, B. Staphylococcus aureus, C. Klebsiella pneumoniae, D. Acinetobacter baumannii, E. Pseudomonas aeruginosa, F Enterobacter cloacae, and H. Klebsiella pneumoniae, plus the common sepsis-associate yeast, G. Candida albicans.

FIG. 7. Relative nanopore sequence outputs for 23 multiplexed, calibrated primer sets in human DNA backgrounds from 0 to 10 μg per PCR reaction.

FIG. 8. Relative nanopore sequence outputs for 23 multiplexed, calibrated primer sets in purified from whole human blood with calibrant inputs from 1×10⁶/mL to 1×10⁴ mL.

FIG. 9. “MotoLyser” Microbial Cell Disruption schematic.

FIG. 10. 3D-Printed “MotoLyser” Microbial Cell Disruption schematic.

FIG. 11. Software Control for MotoLyser device.

FIG. 12. Three-sample MotoLyser Device: A. Base; B. Top and Motor Housing: C. Sliding Latch; D. Bead Agitator; E. Gear Mesh for Bead Agitator; F. Connector pin for Interfacing Bead Agitator and Gear Mesh Through Sealed Bearing; G. Motor Adapter for Meshing to Bead Agitator. Distances indicated in millimeter (mm) and angles in degrees.

### DETAILED DESCRIPTION OF THE INVENTION

Unless defined otherwise, all technical and scientific terms used herein have the same meaning as is commonly understood by one of ordinary skill in the art to which this invention belongs.

As used in the description of the invention and the appended claims, the singular forms “a”, “an” and “the” are used interchangeably and intended to include the plural forms as well and fall within each meaning, unless the context clearly indicates otherwise. Also, as used herein, “and” or “or” refers to and encompasses any and all possible combinations of one or more or two or more of the listed items, as well as the lack of combinations when interpreted in the alternative (“or”).

As used herein. “one or more” is intended to mean “at least one” or all of the listed elements.

Except where noted otherwise, capitalized and non-capitalized forms of all terms fall within each meaning.

Unless otherwise indicated, it is to be understood that all numbers expressing quantities, ratios, and numerical properties of ingredients, reaction conditions, and so forth used in the specification and claims are contemplated to be able to be modified in all instances by the term “about.” As used herein, the term “about” when used before a numerical designation. e.g., temperature, time, amount, concentration, and such other, including a range, indicates approximations which may vary by (+) or (−) 10%, 5% or 1%.

The invention provides methods for broad analysis and quantification of microorganisms and drug-resistance determinants in samples including, but not limited to, liquid blood or lung aspirates.

### Methods of the Invention

The invention provides a method for detecting the presence, identifying, and/or quantifying an amount of an unidentified microbial agent in a sample comprising: (a) mixing a sample with quantified calibrator molecules; (b) isolating nucleic acid in the sample; (c) interrogating and amplifying select nucleic acid sequences with a collection of primer pairs so as to produce double-stranded DNAs, wherein each double-stranded DNA comprises an identifier nucleic acid sequence, a universal amplification target sequence, and a chemical tag; (d) isolating the double-stranded DNAs so produced in step (c) using the chemical tag; (e) determining DNA sequence of the isolated double-stranded DNAs; (f) identifying and quantifying the output of the calibrator molecules added in step (a); (g) organizing DNA sequences so obtained in step (c) to produce a collection of consensus sequences; (h) comparing the collection of consensus sequences against reference sequences so as to detect the presence of an unidentified microbial agent and identify the microbial agent in the sample; and (i) optionally, comparing the signal output levels from each agent identified in the sample to the output levels of the calibrator molecules in order to quantify the genome copy amount of the agent in the original sample.

The sample may be in liquid. The sample may be a liquid sample such as blood or lung aspirate. The sample may be from a subject. The sample may be from the environment.

A “subject” may be a vertebrate, preferably a mammal, and more preferably a human. Mammals include, but are not limited to, farm animals (such as cows, sheep, and goats), sport animals (such as bird, reptile, fish and mammal), pets (such as cats, dogs and horses), primates (such as, monkeys, gorillas and chimpanzees), mice and rats.

In an embodiment of the invention, isolating nucleic acid in the sample of step (b) comprises a pH-dependent nucleic acid binding to and release from a solid support.

In an embodiment of the invention, the nucleic acid binds to the solid support at a lower pH (pH 5-6) and detaches from the solid support at a higher pH (pH 8-9).

In an embodiment of the invention, the nucleic acid is DNA.

The method of the invention may further comprise the step of separating the unidentified microbial agent from the sample prior to step (b). The sample may be a blood sample. The blood sample may be a whole blood sample comprising blood cells. The blood cells may be red blood cells.

In an embodiment of the invention, separating the unidentified microbial agent comprises contacting the blood sample with a starch so that red blood cells shrink in volume and aggregate clearing the blood sample by gravity sedimentation, thereby separating the unidentified microbial agent from the sample.

In an embodiment of the invention, the starch is hetastarch or hydroxy-ethyl starch (HES).

The method of the invention may further comprise the step of disrupting the microbial agent so that the agent fractures, exposes and releases its nucleic acid after step (a) and prior to step (b).

In an embodiment of the invention, disrupting may involve the step of physically breaking up the unidentified microbial agent in an agitator.

In an embodiment of the invention, the agitator comprises directional blades and disrupting beads.

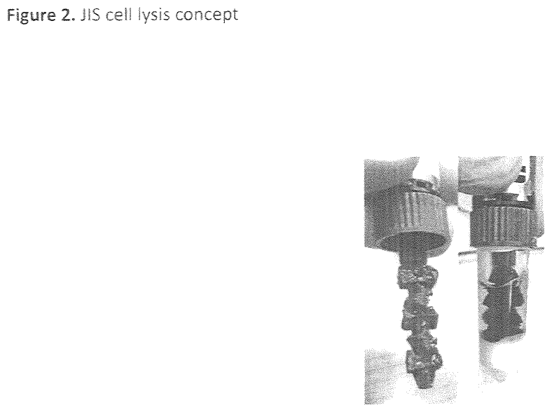

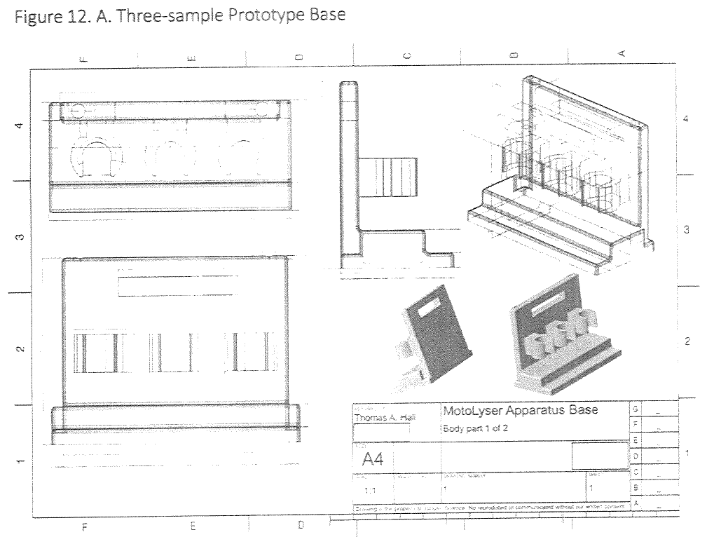

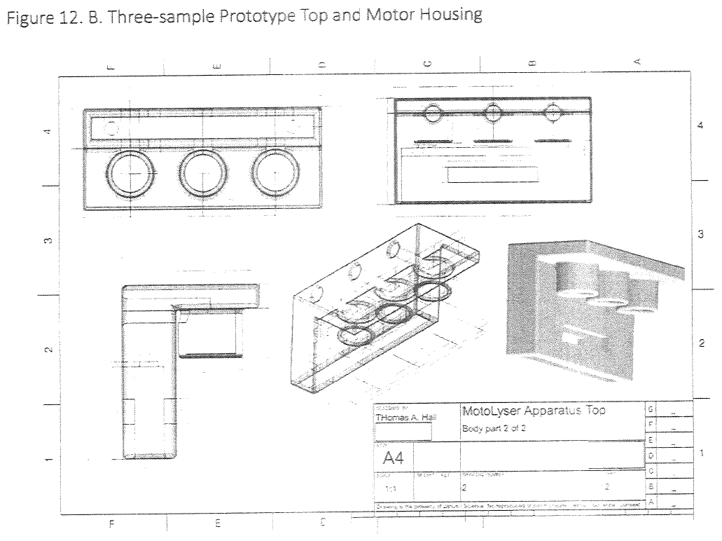

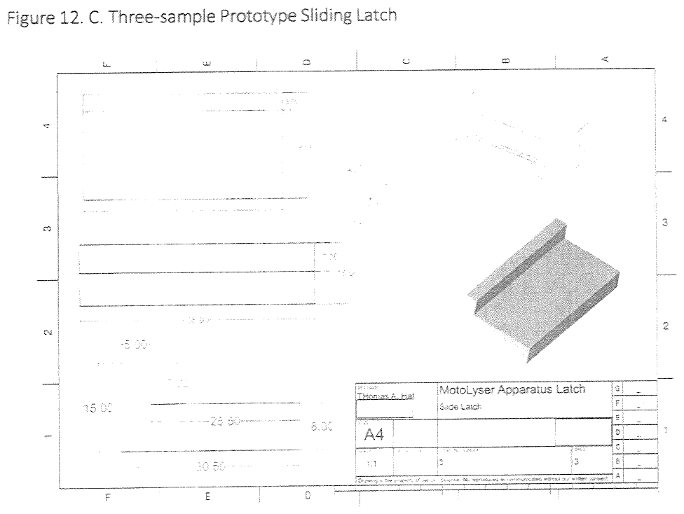

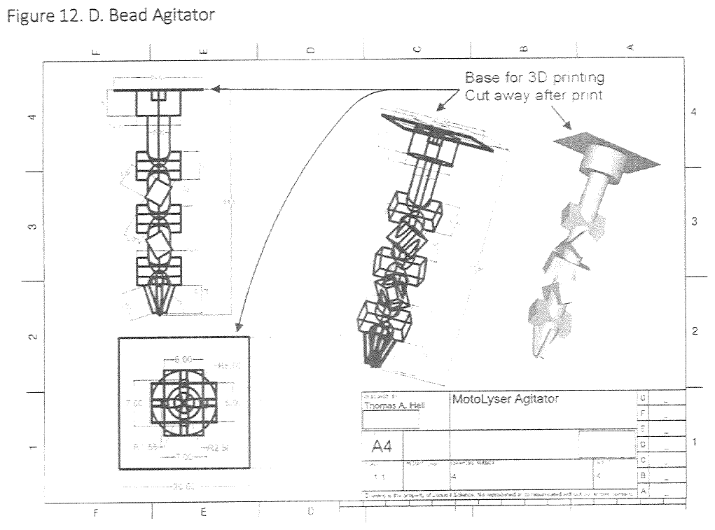

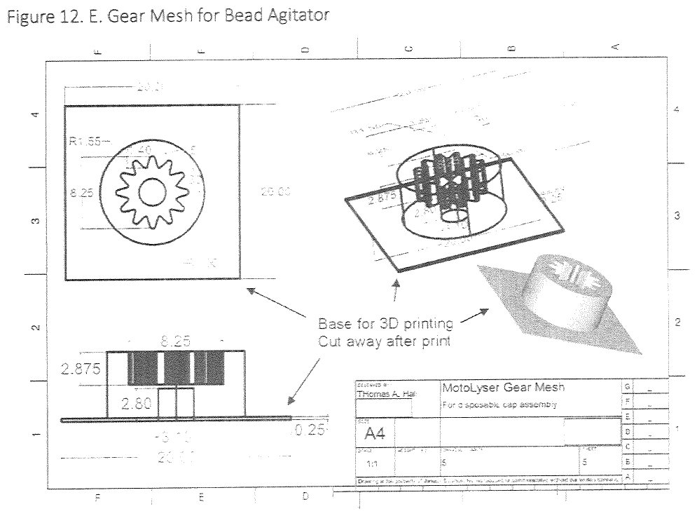

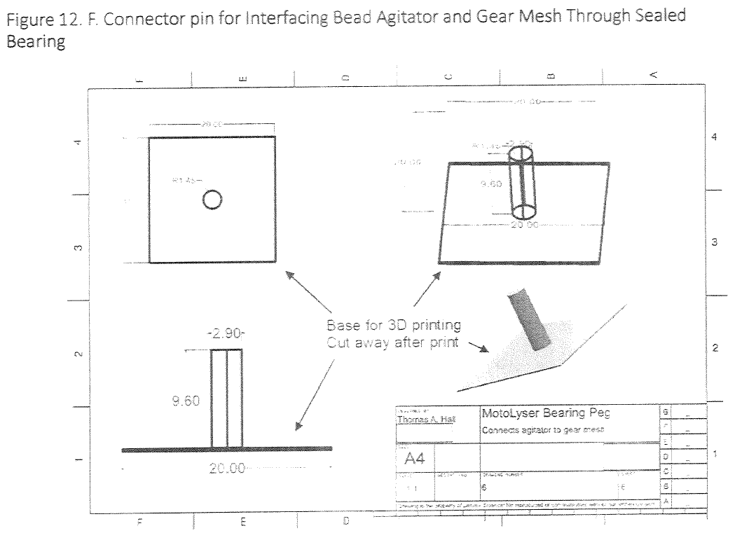

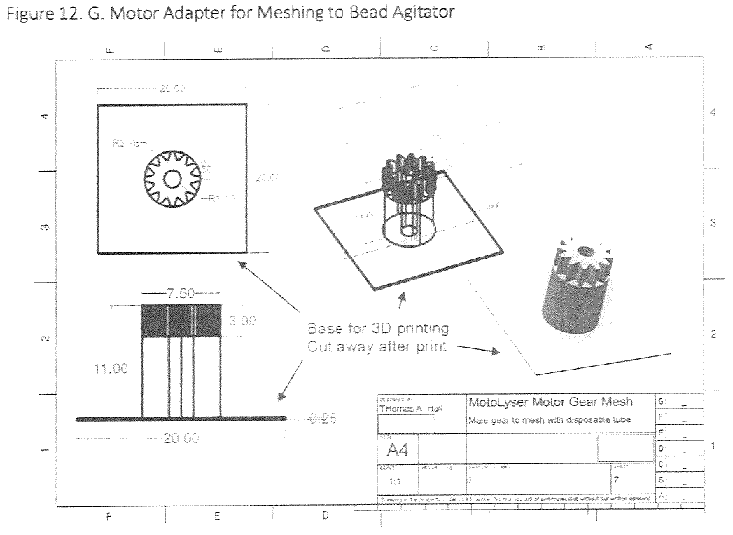

In an embodiment of the invention, the blades are alternating directional blades. In an embodiment, the alternating directional blades are as shown in FIG. 2. In an embodiment, the alternating directional blades have specifications provided in FIG. 12D. In an embodiment, the alternating directional blades are as shown in FIG. 2 with specifications provided in FIG. 12D. In an embodiment, the blades are sterile. In an embodiment, the blades prior to use are free of microbial agent or contaminating nucleic acid.

In an embodiment of the invention, the disrupting beads are yttrium-zirconium beads.

In an embodiment of the invention, the agitator is powered by a motor.

In an embodiment of the invention, the motor is equipped with a toothed gear that meshes to a complementary gear acceptor attached to an agitator blade in a disruptor tube through a sealed bearing in the lid of the tube. The tube may be a glass or plastic tube.

In an embodiment of the invention, the motor produces a cycling routine of short pulses of alternating direction. In an embodiment of the invention, each pulse is about 2 seconds.

In an embodiment of the invention, the motor is controlled by a microcontroller with an automated run mode.

In an embodiment of the invention, the microcontroller is an Arduino Nano microcontroller or equivalent.

In an embodiment of the invention, the microcontroller can be controlled by serial connection to a PC-based controller application. In an embodiment, the PC-based controlled application is written in C#.NET. In an embodiment, the PC-based controlled application is written for Windows OS.

In an embodiment of the invention, the motor is equipped with a toothed gear that meshes to a complementary gear acceptor attached to an agitator blade in a disruptor tube through a sealed bearing in the lid of the tube.

In an embodiment of the invention, the agitator blade is sterile.

In an embodiment of the invention, the tube is plastic.

In an embodiment of the invention, the motor is equipped with a toothed gear that meshes to a complementary gear acceptor attached to a sterile agitator blade in a sterile plastic tube through a sealed bearing in the lid of the tube.

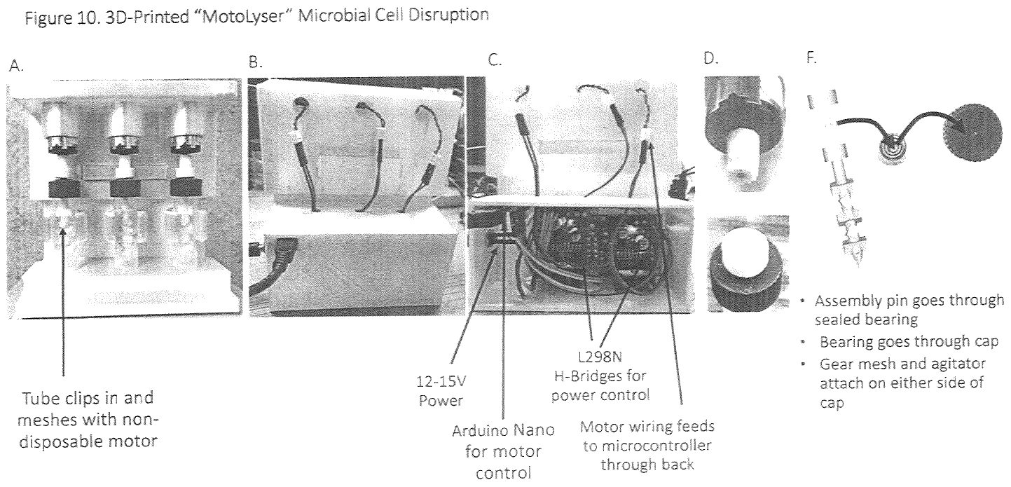

In an embodiment of the invention, the toothed gear comprises a concentric ring of v-shaped teeth on a cylindrical rod or spindle. In an embodiment, the toothed gear comprises anywhere from 8 to 16 teeth. In a preferred embodiment, the toothed gear comprises 12 teeth as shown in FIG. 10D top panel. In a preferred embodiment, the toothed gear comprising 12 teeth has a specification with dimensions or relative dimensions as shown in FIG. 12G.

In an embodiment of the invention, the complementary gear acceptor on the lid of the tube meshes with the toothed gear. In an embodiment, the complementary gear acceptor meshes with a toothed gear chosen from a range of 8-16 teeth. In a preferred embodiment, the complementary gear acceptor meshes with a 12-teeth gear. In a preferred embodiment, the complementary gear acceptor meshes with a 12-teeth gear as shown in FIG. 12E. In a preferred embodiment, the complementary gear acceptor meshing with 12-teeth gear has a specification with dimensions or relative dimensions as shown in FIG. 12E.

In an embodiment of the invention, the motor is attached to a solid support which provides hand-free disruption of the microbial agent.

In an embodiment of the invention, the motor is one of two or more motors attached to a solid support which provides hand-free disruption of the microbial agent and optionally multiple sample disruptions simultaneously.

In an embodiment of the invention, the solid support further comprises a port for motor wiring, slide-in locking latch, vertical tube support, a base stand, and a top support for motor mount.

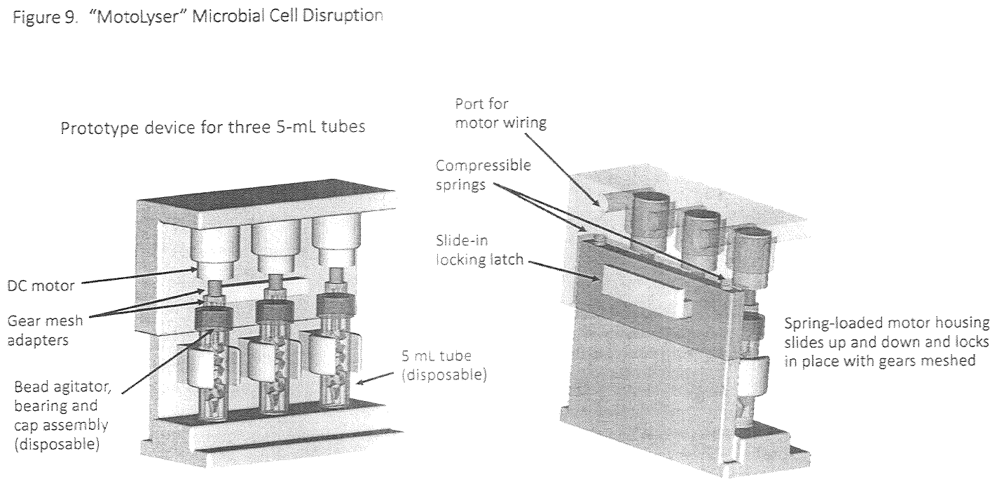

In an embodiment of the invention, the solid support allows simultaneous processing of samples in 3 or more disruptor tubes. In an embodiment, the solid support fully or partially assembled with or without disruptor tubes is as shown in FIGS. 9 to 12. In an embodiment, the solid support has a specification with dimensions or relative dimensions as shown in FIGS. 9 to 12.

In an embodiment of the invention, the two or more motors attached to a solid support are the same type of motor. In an embodiment of the invention, the two or more motors are controlled by a single microcontroller. In a separate embodiment of the invention, the two or more motors are controlled by one or more separate microcontrollers or a dedicated microcontroller for each motor.

In an embodiment of the invention, the solid support comprises a spring-loaded housing fixed to each motor, wherein the spring-loaded housing permit convenient securing and meshing of the motor to the complementary gear acceptor attached to the lid of the disruptor tube.

In an embodiment of the invention, the disruptor tube comprises the sample in contact with the agitator.

In an embodiment of the invention, multiple motors are equipped in a device that allows multiple sample disruptions simultaneously.

In an embodiment of the invention, the motors are fixed to a spring-loaded housing in a device that allows convenient securing and meshing of the motors to the disruptor tubes.

In an embodiment of the invention, each primer pair comprises a forward primer and a reverse primer for interrogating and amplifying a select nucleic acid sequence. The select nucleic acid sequence may comprise an identifier nucleic acid sequence. The identifier nucleic acid sequence may be a microbial nucleic acid sequence. The microbial nucleic acid targets may be homologous to any of sequences specified in Table 1 and Table 4 and combination thereof.

In a preferred embodiment, the microbial nucleic acid targets may be homologous to any of sequences specified in Table 4 and combination thereof.

In an embodiment of the invention, each primer comprises a specificity target region and a universal amplification region, and optionally a barcode unique to the primer. In an embodiment, the specificity target region directs the primer to a primer binding site in a target nucleic acid sequence. In an embodiment, the universal amplification region serves as potential primer binding site for further or optional amplification of an amplified DNA product at step (e) of claim 1.

In a preferred embodiment of the invention, each primer consists of a specificity-targeting region and a universal amplification target region.

In an embodiment of the invention, the primer comprises the universal amplification region upstream or 5′ of the specificity target region. In an embodiment, the primer comprises the universal amplification region upstream or 5′ of the specificity target region and the optional barcode between the universal amplification region and the specificity target region.

In an embodiment of the invention, one primer of each primer pair comprises a chemical tag. In an embodiment, the primer comprises a chemical tag at 5′ end of the primer. In a preferred embodiment, the primer comprises a chemical tag at 5′ end of the primer when the chemical tag is present.

In an embodiment of the invention, one primer lacks a chemical tag.

In an embodiment of the invention, each primer may comprise a unique bar code.

In an embodiment of the invention, each primer pair comprises a chemical tag in either reverse or forward primer. In an embodiment of the invention, each primer pair comprises a chemical tag in the forward primer. In an embodiment of the invention, each primer pair comprises a chemical tag in the reverse primer. In an embodiment of the invention, each primer pair comprises a chemical tag in either reverse or forward primer but not both primers.

In an embodiment of the invention, each primer comprises a specificity target region and a universal amplification region and each primer of a primer pair comprises a chemical tag at 5′ end of either a reverse or forward primer.

In an embodiment of the invention, each primer comprises a specificity target region, a barcode and a universal amplification region and each primer of a primer pair comprises a chemical tag at 5′ end of either reverse or forward primer.

In an embodiment of the invention, the chemical tag is present only on one but not both primers of the primer pair.

In an embodiment of the invention, each primer pair additionally comprises a first unique universal sequence on the 5′ end of one of two primers in the primer pair and a second unique universal sequence on the 5′ end of the remaining primer in the primer pair. In an embodiment, the one primer may be a forward primer and the other primer may be a reverse primer. In a separate embodiment, the one primer may be a reverse primer and the other primer may be a forward primer. In an embodiment of the invention, a collection of primer pairs used to interrogate and amplify select nucleic acid sequences comprises primer pairs wherein each primer pair comprises a first unique universal sequence in one primer and a second unique universal sequence in the other primer.

In an embodiment of the invention, the universal amplification region of each primer comprises a first unique universal sequence and a second unique universal sequence, wherein one primer of a primer pair comprises the first unique universal sequence and the other primer of the primer pair comprises the second unique universal sequence and wherein the primer pairs in the collection of primer pairs comprises the first unique universal sequence for one primer and the second unique universal sequence for the other primer, such that the first and second unique universal sequences are shared across all primer pairs in the collection of primers.

In an embodiment of the invention, the primer is any of the primers as provided in Table 2 or Table 5, wherein location of optional biotin tag at 5′ end of one of the pair of primers is indicated as/5Biosg/.

In a preferred embodiment of the invention, the primer is any of the primers as provided in Table 5, wherein location of optional biotin tag at 5′ end of one of the pair of primers is indicated as/5Biosg/.

In an embodiment of the invention, the primer pair comprises a tag at 5′ end of one of the pair of primers. In an embodiment of the invention, the tag is biotin. In an embodiment of the invention, the tag is a chemical or peptide tag. In an embodiment of the invention, the tag is (His)₆. In a separate embodiment of the invention, the tag is an epitope tag. In an embodiment of the invention, the epitope tag is selected from the group consisting of myc. HA, V5, and FLAG.

In an embodiment of the invention, interrogating and amplifying select nucleic acid sequences comprise polymerase chain reaction (PCR). The polymerase chain reaction may be multiplex PCR comprising nucleic acid and the collection of primer pairs in a single reaction vessel.

In an embodiment of the invention, the collection of primer pairs comprises at least 17 forward-reverse primer combinations or at least 37 distinct primers in a single reaction vessel.

In an embodiment of the invention, the collection of primer pairs is 22 forward-reverse primer combinations or 37 distinct primers in a single reaction vessel.

In a preferred embodiment of the invention, the collection of primer pairs comprises at least 24 forward-reverse primer combinations or at least 50 distinct primers in a single reaction vessel.

Examples of the primer pairs may include, but are not limited to, any of the primer pairs as provided in Table 2 or Table 5, wherein location of optional biotin tag at 5′ end of one of the pair of primers is indicated as/5Biosg/. In an embodiment, the primer pairs are selected from the group consisting of any of the primer pairs as provided in Table 2 or Table 5.

In an embodiment of the invention, one or more calibrator molecule(s) is added to the sample in a known quantity prior to mixing the sample with quantified calibrator molecules. The mixing a sample with quantified calibrator molecules may occur in a liquid phase. In an embodiment, the calibrator molecule is a nucleic acid. The nucleic acid may be DNA The DNA can be double-stranded. The calibrator molecule can be considered a synthetic calibrator molecule.

In an embodiment of the invention, each primer pair in a multiplex has a specific calibrator molecule as a PCR amplification target.

In an embodiment of the invention, the calibrator molecule is a synthetic calibrator molecule comprising primer binding sites for a pair of primers as provided in Tables 2 and 5 separated by an intervening sequence, wherein the primer binding sites comprise the same or similar sequences as primer binding sites found within Reference Sequence at Coordinates provided in Tables 1 and 4.

In an embodiment of the invention, the intervening sequence in the synthetic calibrator comprises a unique sequence that is distinct and unambiguously distinguishable from bacterial, fungal, or antimicrobial resistance gene targets referred to in Table 1 and 4.

In an embodiment of the invention, the intervening sequence in the synthetic calibrator has or comprises a nucleotide composition of a sequence found between primer binding sites for the sequence or its complement associated with the Coordinates for target organisms referred to in Table 1 or 4.

In an embodiment of the invention, the intervening sequence in the synthetic calibrator comprises a nucleic acid sequence derived from a sequence found between primer binding sites for the sequence or its complement associated with the Coordinates for target organisms referred to in Table 1 or 4.

In an embodiment of the invention, the intervening sequence in the synthetic calibrator comprises a random ordering or shuffling of a sequence found between a primer pair for the targets referred to in Table 1 or 4 so as to produce a unique and unambiguously distinguishable sequence.

In an embodiment of the invention, the intervening sequence in the synthetic calibrator is synthetic and not naturally occurring.

In an embodiment of the invention, the calibrator molecule is synthetic and not naturally occurring.

In an embodiment of the invention, the calibrator molecule is a nucleic acid with any of the sequences and their complements as provided in Table 6.

In an embodiment of the invention, amplifying select nucleic acid sequences with a primer pair produces same size DNA or same DNA length for the calibrator molecule and targets referred to in Table 1 or 4 when amplified by the same primer pair.

In an embodiment of the invention, the calibrator molecule competes for amplification with the bacterial, fungal or antimicrobial resistance DNA targets in the sample.

In an embodiment of the invention, comparison of the amplified bacterial, fungal or antimicrobial resistance DNA targets with the amplified calibrator molecule provides a quantitative estimate of amount of pathogen DNA or a particular microbial agent in the sample.

In an embodiment of the invention, the sample additionally comprises a calibrator molecule for each primer pair in the collection of primer pairs of the methods of the invention.

In an embodiment of the invention, the calibrator molecule for each primer pair is as specified in Table 6, such that the collection of primer pairs specifies a group of calibrator molecules of Table 6.

In an embodiment of the invention, synthetic calibrator nucleic acids are added to the sample in known quantities prior to purification of nucleic acids from the liquid sample.

In an embodiment of the invention, these synthetic calibrator nucleic acids are double stranded DNA molecules.

In an embodiment of the invention, the synthetic calibrator DNA molecules contain the same primer target regions as those in the primers depicted in Table 2.

In a preferred embodiment of the invention, the synthetic calibrator DNA molecules contain the same primer target regions as those in the primers depicted in Table 5.

In an embodiment of the invention, the primer target regions in these synthetic calibrator sequences are placed on either side of a unique sequence that is distinct from all known nucleic acid sequences and is unambiguously differentiable from the bacterial, fungal, or antimicrobial resistance gene targets referred to in Tables 1 and 4.

In an embodiment of the invention, the amplified region of the synthetic calibrator targets has a nucleotide composition based upon the targets referred to in Tables 1 and 4, but in a randomly-scrambled sequence order.

In an embodiment of the invention, the synthetic calibrator molecules compete directly with the bacterial, fungal of antimicrobial resistance DNA targets in the sample such that a comparison of the observed output levels of each provides a quantitative estimate of the amount of pathogen target DNA in the original sample.

In an embodiment of the invention, the calibrator DNA sequences are embedded in a plasmid construct replicated in a bacterium such as E. coli.

In an embodiment of the invention, the calibrator DNA sequences are purified from PCR-amplified products amplified from a small quantity of the calibrator plasmid construct using primer pairs common to all constructs.

In an embodiment of the invention, there exists a calibrator DNA target for all primer sets in the first PCR reaction and calibrators are mixed together into one multiplexed mixture and added to a sample prior to nucleic acid purification.

In an embodiment of the invention, the synthetic calibrator sequences may include, but are not limited to, any of the synthetic calibrator sequences as provided in Table 6.

In an embodiment of the invention, after isolating nucleic acid from the sample but prior to interrogating and amplifying select nucleic acid sequences, a solution comprising isolated nucleic acid and calibrator molecules additionally comprises a defined amount of internal process control sequence.

In an embodiment of the invention, the internal process control sequence is a double stranded DNA.

In an embodiment of the invention, the internal process control sequence is synthetic and not naturally occurring.

In an embodiment of the invention, the internal process control sequence comprises the following sequence or a portion thereof:

CCTGCCCTTCGTACCCGGATCGCTCGCCCGGCCGGCTGATCTTGGCTCG
GCCCGCGGATGATCCCGAACTCGTTTTAACAGAGACGCTGTGGGCGGCC
GGATATATCCGACGCAAACAGTGGTCCCGTGGCCTTTTAGTAACAAAGA
TAAACCGGCCGACGCGGCGAGGGTCATCGTAGTTCACCTCTGTAGCCGG
ATACCGAGAGGGCGGGGCCACTCCCGATTGTGACTTCGGCTCCGAGCGA
GTCCACCTCGTGCGGGGTGCAGGACCTCAGAATCACGGCGGGCTGGGCA
ATAGCCTCGCGCTGTCAAAGTGAGCCGCACTAACAACGACGGTTAGCGT
AGTGCATCCTTCCTTCGGGACAAAATCATACTAAGTTCGTCTTACACGA
CAAAATACGACAGGGAACGCCTTCACAGGACGCCTCGTTTGTAGGTCTG
GGGCCCGGCCTGGGGCGGACTGCGATCAGGCCAGGTCGACTCTGCCTGA
CCGGATAGGGCCCCGTTGTGAGATCAAAGCGGGATTCTCGAGATGCTCT
TAGTCATGGGTAATGAACCCCTCCTGCCCCCAGCAGGATGAGTAGCGGG
ACCGCAACCGGTCCATTGAACTCGTCTAGCGCACTAGACTTGCGTCCCT
AGCGCCCGATAGGCGGTAAACCATAACCAACCCCTGCTGGACGAGGCGG
TCGCGGAACGCGCCTGCCCAAGATTTACACGGGTGTAAGTTCTCGGAAT
TAGCACTTTTTTGACGGGCGAAGTCACGAGCGCTTTGTACGCCTCTCGA
TCGAGCCGGGCTGGGACGCCCGTCGGCCGGTGGTAATCCTTACGGAGGT
CATGCGCCTTCACTGGGCCCTGACTTCCTGGGAGCTTCGTCTTGTGAAC
CTAGGGAGCAATGTGAACCGTGCCTCATCGGAGCGGCCTACAGCAGGCT
CGACCTGGGCGTAACGGACGCCGACATAGGATTCGCCTTGCAATACCCT
GCGGAAGCTTCGGCATCGT.

In an embodiment of the invention, the DNA is about 100 bp to 1,000 bp long.

In an embodiment of the invention, the DNA is about 1,000 bp or longer.

In an embodiment of the invention, an additional internal process control sequence is added directly to the first PCR in a known quantity.

In an embodiment of the invention, the synthetic process control is a 1000-base pair, double stranded DNA molecule with the following sequence:

CCTGCCCTTCGTACCCGGATCGCTCGCCCGGCCGGCTGATCTTGGCTCG
GCCCGCGGATGATCCCGAACTCGTTTTAACAGAGACGCTGTGGGCGGCC
GGATATATCCGACGCAAACAGTGGTCCCGTGGCCTTTTAGTAACAAAGA
TAAACCGGCCGACGCGGCGAGGGTCATCGTAGTTCACCTCTGTAGCCGG
ATACCGAGAGGGCGGGGCCACTCCCGATTGTGACTTCGGCTCCGAGCGA
GTCCACCTCGTGCGGGGTGCAGGACCTCAGAATCACGGCGGGCTGGGCA
ATAGCCTCGCGCTGTCAAAGTGAGCCGCACTAACAACGACGGTTAGCGT
AGTGCATCCTTCCTTCGGGACAAAATCATACTAAGTTCGTCTTACACGA
CAAAATACGACAGGGAACGCCTTCACAGGACGCCTCGTTTGTAGGTCTG
GGGCCCGGCCTGGGGCGGACTGCGATCAGGCCAGGTCGACTCTGCCTGA
CCGGATAGGGCCCCGTTGTGAGATCAAAGCGGGATTCTCGAGATGCTCT
TAGTCATGGGTAATGAACCCCTCCTGCCCCCAGCAGGATGAGTAGCGGG
ACCGCAACCGGTCCATTGAACTCGTCTAGCGCACTAGACTTGCGTCCCT
AGCGCCCGATAGGCGGTAAACCATAACCAACCCCTGCTGGACGAGGCGG
TCGCGGAACGCGCCTGCCCAAGATTTACACGGGTGTAAGTTCTCGGAAT
TAGCACTTTTTTGACGGGCGAAGTCACGAGCGCTTTGTACGCCTCTCGA
TCGAGCCGGGCTGGGACGCCCGTCGGCCGGTGGTAATCCTTACGGAGGT
CATGCGCCTTCACTGGGCCCTGACTTCCTGGGAGCTTCGTCTTGTGAAC
CTAGGGAGCAATGTGAACCGTGCCTCATCGGAGCGGCCTACAGCAGGCT
CGACCTGGGCGTAACGGACGCCGACATAGGATTCGCCTTGCAATACCCT
GCGGAAGCTTCGGCATCGT.

In an embodiment of the invention, the internal process control is embedded in a bacterial plasmid construct.

In an embodiment of the invention, the polymerase chain reaction is carried out in the early exponential phase of the reaction, or about 10-25 cycles.

In an embodiment of the invention, the polymerase chain reaction is performed in a thermal cycler.

In an embodiment of the invention, the polymerase chain reaction is performed isothermally.

In an embodiment of the invention, the polymerase chain reaction is performed in a fluidic chip.

In an embodiment of the invention, the polymerase chain reaction is performed in the presence of betaine.

In an embodiment of the invention, the double-stranded DNA so amplified comprises a forward and a reverse DNA strand which are complementary, wherein each strand comprises an internal identifier nucleic acid sequence.

In an embodiment of the invention, the double-stranded DNA so amplified comprises a forward and a reverse DNA strand which are complementary, wherein each strand has an identifier nucleic acid sequence optionally between a first barcode on 5′ side and a second barcode on 3′ side, so that each strand comprises an internal identifier nucleic acid sequence and optionally a double barcode.

In an embodiment of the invention, the strand additionally comprises a first unique universal sequence or its complement.

In an embodiment of the invention, the first unique universal sequence or its complement is upstream of the first barcode when the barcode is present.

In an embodiment of the invention, the strand additionally comprises a second unique universal sequence or its complement.

In an embodiment of the invention, the second unique universal sequence or its complement is downstream of the second barcode when the barcode is present.

In an embodiment of the invention, each strand comprises a first unique universal sequence or its complement upstream of the first barcode and a second unique universal sequence or its complement downstream of the second barcode, when the barcodes are present.

In an embodiment of the invention, the double-stranded DNA additionally comprises a chemical tag.

In an embodiment of the invention, the chemical tag is associated with either the forward or reverse DNA strand.

In an embodiment of the invention, the chemical tag is associated with either the forward or reverse DNA strand but not both.

In an embodiment of the invention, the chemical tag is biotin.

In an embodiment of the invention, following the step of interrogating and amplifying select nucleic acid sequences with a collection of primer pairs and prior to step of isolating the double-stranded DNAs so produced using the chemical tag, the method additionally comprises a step of contacting sample liquid comprising amplified DNAs and unused single-stranded primers with exonuclease I so as to fragment unused single-stranded primers.

In an embodiment of the invention, isolating the double-stranded DNAs comprises contacting the double-stranded DNA so produced with immobilized streptavidin or avidin to a solid surface.

In an embodiment of the invention, the solid surface is a surface on a bead or surface in a flow cell.

In an embodiment of the invention, the step of determining DNA sequence of the isolated double-stranded DNA comprises an optional step of amplifying the double-stranded DNAs to further enrich the double-stranded DNAs.

In an embodiment of the invention, amplifying the double-stranded DNAs comprises a primer pair comprising a forward primer comprising sequences complementary to the unique universal sequence at the 3′ end of each strand of the double-stranded DNA, wherein each strand comprises either 1^{st} or 2^{nd} unique universal sequence at its 3′ end and serves as a universal amplification target sequence.

In an embodiment of the invention, amplifying the double-stranded DNAs comprises a primer pair comprising a forward primer comprising the unique universal sequence at the 5′ end of the double-stranded DNA and a reverse primer comprising a sequence complementary to the second unique universal sequence at the 3′ end of the double-stranded DNAs.

In an embodiment of the invention, universally amplifying the double-stranded DNAs further comprises polymerase chain reaction.

In an embodiment of the invention, determining DNA sequence of the isolated double-stranded DNA comprises single molecule DNA sequencing.

In an embodiment of the invention, the single molecule DNA sequencing comprises:
attaching an adapter DNA to an end of the double-stranded DNA or amplified DNA so as to permit interaction with a nanopore;
ii) contacting the double-stranded DNAs attached to an adapter DNA with a nanopore;
iii) recording current or change in current as one of the two strands transverses the nanopore; and
iv) correlating current or change in current over time to passage of a particular nucleic acid base through the nanopore, thereby performing single molecule DNA sequencing.

In an embodiment of the invention, single molecule DNA sequencing is performed in a nanopore flow cell. Merely by way of example, the nanopore flow cell may be an Oxford Nanopore Technologies' MinION flow cell or its equivalent.

In an embodiment of the invention, the step of organizing DNA sequences so obtained may comprise:
v) organizing all DNA sequences by recognition of the two primer-target+universal primer sequences at the 5′ end and 3′ end of each DNA sequence so as to obtain groups obtained from the same primer sets or their complements;
vi) aligning all members of the group by forward or reverse sequence, and if necessary converting forward to reverse sequence or vice versa; and
vii) producing a consensus sequence for each group of aligned forward or reverse sequences.

In an embodiment of the invention, organizing DNA sequences so obtained comprises:
sorting all DNA sequences into groups according to primer target sites at their 5′ end and 3′ ends so as to obtain groups with members generated by the same primer pair or their complements;
vi) aligning all members of the group by forward or reverse sequence, and if necessary converting forward to reverse sequence or vice versa; and
vii) producing a consensus sequence for each group of aligned forward or reverse sequences.

In an embodiment of the invention, comparing the collection of consensus sequences against reference sequences comprises:
viii) matching a consensus sequence against identifier nucleic acid sequences so as to identify the presence of an identifier nucleic acid sequence and all possible microbial agents associated with the identifier nucleic acid sequence;
ix) repeating step (viii) for remaining consensus sequences;
x) analyzing possible microbial agents so identified by steps (viii) and (ix) for each consensus sequence for overlapping sets of microbial agents so as to detect the presence of an unidentified microbial agent and identify the microbial agent in the sample from the subject.

In an embodiment of the invention, the identifier nucleic acid sequence is a portion of a sequence. Examples of the sequence may include, but are not limited to, bacterial 16S rDNA, 23S rDNA, rpoB, tufB, valS, fungal/yeast mitochondrial SSU rDNA, fungal/yeast 25S rDNA, KPC carbapenemase gene, vanA, vanB and mecA.

In an embodiment of the invention, the identifier nucleic acid sequence is associated with bacterial domain, bacterial phylum, bacterial class, bacterial order, bacterial family, bacterial genus, bacterial species, bacterial subtype bacterial strain, fungal phylum, fungal class, fungal order, fungal family, fungal genus or fungal species.

In an embodiment of the invention, the bacterial subtype is selected on the basis of drug resistance marker.

In an embodiment of the invention, the drug resistance marker is any of, but not limited to, carbapenemase gene, KPC carbapenemase gene, vanA, vanB and mecA or a combination thereof.

In an embodiment of the invention, the identifier nucleic acid sequence is a portion of a sequence from a virus.

In an embodiment of the invention, the unidentified microbial agent is a virus, bacterium, parasite and/or a fungus. For example, the bacterium and/or fungus may be further classified as belonging to a particular domain, kingdom, phylum, class, order, family, genus, species and/or subtype. In accord with the invention, the subtype may be discriminated on the basis of a drug resistance marker. Merely by way of example, the drug resistance marker may be selected from the group of KPC, vanA, vanB and mecA. In an embodiment of the invention, the bacterium and/or fungus may be classified as belonging to a kingdom, phylum, family, genus and species as provided in Table 7.

In an embodiment of the invention, the subject is infected with a microbial agent or suspected to be infected with a microbial agent.

Examples of the microbial agent may include, but are not limited to, virus, bacterium, parasite and fungus. For example, the bacterium may be a Gram-positive or Gram-negative bacterium.

In an embodiment of the invention, the microbial agent is from the genus selected from the group consisting of Acetoanaerobium, Acholeplasma, Achromobacter Acidimicrobium, Acidiphilium, Acidovorax, Acinetobacter, Actinobacillus, Actinomyces, Aerococcus, Aeromonas, Aliivibrio, Anaplasma, Anoxybacillus, Aspergillus, Bacillus, Bacteroides, Bartonella, Bifidobacterium, Blattabacterium, Bordetella, Borrelia, Borreliella, Brachyspira, Brevibacterium, Burkholderia, Campylobacter, Candida, Capnocytophaga, Caulobacter, Chlamydia, Citrobacter, Clavispora, Clostridioides, Clostridium, Corynebacterium, Coxiella, Cunninghamella, Dechloromonas, Desulfitobacterium, Edwardsiella, Ehrlichia, Enterobacter, Enterococcus, Erwinia, Escherichia, Flavobacterium, Francisella, Fusarium, Fusobacterium, Gordonia, Haemophilus, Helicobacter, Histoplasma, Hyphopichia, Klebsiella, Kluyvera, Kluyveromyces, Lacticaseibacillus, Lactobacillus, Lactococcus, Legionella, Lentilactobacillus, Leptospira, Leuconostoc, Listeria, Loigolactobacillus, Malassezia, Metabacillus, Microbacterium, Moraxella, Mucor, Mycobacterium, Mycoplasma, Neisseria, Nocardia, Paenibacillus, Paraburkholderia, Pasteurella, Penicillium, Phocaeicola, Pneumocystis, Porphyromonas, Prevotella, Proteus, Pseudoalteromonas, Pseudomonas, Ralstonia, Rhodococcus, Rickettsia, Rothia, Saccharomyces, Salmonella, Scedosporium, Schizosaccharomyces, Serratia, Shewanella, Shigella, Sphingomonas, Spiroplasma, Staphylococcus, Streptococcus, Teunomyces, Treponema, Vibrio, Xanthomonas, Zobellella, and Zymomonas. In an embodiment of the invention, the microbial agent is a bacterium belonging to a genus selected from the group consisting of Acetoanaerobium, Acholeplasma, Achromobacter, Acidimicrobium, Acidiphilium, Acidovorax, Acinetobacter, Actinobacillus, Actinomyces, Aerococcus, Aeromonas, Aliivibrio, Anaplasma, Anoxybacillus, Bacillus, Bacteroides, Bartonella, Bifidobacterium, Blattabacterium, Bordetella, Borrelia, Borreliella, Brachyspira, Brevibacterium, Burkholderia, Campylobacter, Capnocytophaga, Caulobacter, Chlamydia, Citrobacter, Clostridioides, Clostridium, Corynebacterium, Coxiello, Dechloromonas, Desulfitobacterium, Edwardsiella, Ehrlichia, Enterobacter, Enterococcus, Erwinia, Escherichia, Flavobacterium, Francisella, Fusobacterium, Gordonia, Haemophilus, Helicobacter, Klebsiella, Kluyvera, Locticaseibacillus, Lactobacillus, Lactococcus, Legionella, Lentilactobacillus, Leptospira, Leuconostoc, Listeria, Loigolactobacillus, Metabacillus, Microbacterium, Moraxella, Mycobacterium, Mycoplasma, Neisseria, Nocardia, Poenibacillus, Paraburkholderia, Pasteurella, Phocaeicola, Porphyromonas; Prevotella. Proteus, Pseudoalteromonas, Pseudomonas, Ralstonia, Rhodococcus, Rickettsia, Rothia, Salmonella, Serratia, Shewanella. Shigella, Sphingomonas, Spiroplasma, Staphylococcus, Streptococcus, Treponema, Vibrio, Xanthomonas, Zobellella, and Zymomonus. In an embodiment of the invention, the microbial agent is a fungus belonging to the genus selected from the group consisting of Aspergillus, Candida, Clavispora, Cunninghamella, Fusarium, Histoplasma, Hyphopichia, Kluyveromyces, Malassezia, Mucor, Penicillium, Pneumocystis, Saccharomyces, Scedosporium, Schizosaccharomyces, and Teunomyces. In an embodiment of the invention, the microbial agent is from the genus selected from the group consisting of Enterococcus, Staphylococcus, Klebsiella, Acinetobacter, Pseudomonas, Enterobacter, and Candida.

In an embodiment of the invention, the microbial agent is from the species selected from the group consisting of Acetoanaerobium sticklandii, Acholeplasma oculi, Achromobacter denitrificans, Acidimicrobium ferrooxidans, Acidiphilium multivorum, Acidovorax citrulli, Acinetobacter baumannii, Acinetobacter calcoaceticus, Acinetobacter guillouiae, Acinetobacter haemolyticus, Acinetobacter lactucae, Acinetobacter schindleri, Actinobacillus pleuropneumoniae, Actinobacillus succinogenes, Actinomyces slackit, Actinomyces viscosus, Aerococcus urinae, Aeromonas salmonicida, Aeromonas veronii, Anaplasma ovis, Anoxybacillus flavithermus, Aspergillus clavatus, Aspergillus fumigatus, Aspergillus niger, Aspergillus oryzae, Aspergillus terreus, Bacillus anthracis, Bacillus licheniformis, Bacillus litoralis, Bacillus megaterivon, Bacillus mycoides, Bacillus subtilis, Bacillus thuringiensis, Bacteroides caccae, Bacteroides fragilis, Bacteroides thetaiotaomicron, Bacteroides vulgatus, Bartonella henselae, Bartonella quintana, Bartonella vinsonii, Bifidobacterium adolescentis, Bifidobacterium bifidum, Bifidobacterium gallinarum, Blattabacterium clevelandi, Bordetella bronchialis, Bordetella bronchiseptica, Bordetella parapertussis, Bordetella pertussis, Borrelia hermsii, Borreliella burgdorferi, Brachyspira murdochii, Brevibacterium aurantiacum, Burkholderia ambifaria, Burkholderia contaminans, Burkholderia mallei, Burkholderia pseudomallei, Burkholderia thailandensis, Campylobacter coll, Campylobacter jejuni, Campylobacter lari, Campylobacter ureolyticus, Candida albicans, Candida auris, Candida blattae, Candida castellii, Candida dubliniensis, Candida glabrata, Candida intermedia, Candida metapsilosis, Candida orthopsilosis, Candida parapsilosis, Candida rhagii, Candida sake, Candida tropicalis, Capnocytophaga gingivalis, Caulobacter mirabilis, Chlamydia gallinacean, Chlamydia pneumoniae, Chlamydia psittaci, Chlamydia trachomatis, Citrobacter braakii, Citrobacter freundii, Citrobacter pasteurii, Citrobacter youngae, Clostridioides difficile, Clostridium acetobutyliceum, Clostridium botulinum, Clostridium perfringens, Clostridium sporogenes, Corynebacterium diphtheriae, Corynebacterium flavescens, Corynebacterium glutamicum, Corynebacterium pseudotuberculosis, Corynebacterium simulans, Corynebacterium ulcerans, Coxiella burnetii, Cunninghamella bertholletiae, Dechloromonas aromatica, Desulfitobacterium hafniense, Edwardsiella tarda, Ehrlichia chaffeensis, Enterobacter bugandensis, Enterobacter cloacae, Enterobacter ludwigii, Enterococcus faecalis, Enterococcus faecium, Enterococcus gallinarum, Erwinia persicina, Escherichia coli, Escherichia fergusonii, Flavobacterium branchiophilum, Flavobacterium indicum, Flavobacterium johnsoniae, Flavobacterium pallidum, Francisella noatunensis, Francisella tularensis, Fusarium oxysporum, Fusarium secorum, Fusarium verrucosum, Fusobacterium nucleatum, Fusobacterium ulcerans, Gordonia bronchialis, Haemophilus influenzae, Haemophilus parainfluenzae, Helicobacter pylori, Histoplasma capsulatum, Klebsiella oxytoca, Klebsiella pneumoniae, Klebsiella variicola, Kluryvera intermedia, Kluyveromyces lactis, Kluyveromyces nonfermentans, Lactobacillus acidophilus, Lactobacillus backit, Lactobacillus buchneri, Lactobacillus delbrueckii, Lactobacillus johnsonii, Lactobacillus rhumnosus, Lactococcus lactis, Legionella israelensis, Legionella pneumophila, Leptospira interrogans, Leptospira santarosai, Leuconostoc kimchi, Listeria innocua, Listeria monocytogenes, Malassezia dermatis, Malassezia furfur, Malassezia pachydermatis, Microbacterium hominis, Moraxella catarrhalis, Moraxella cuniculi, Mucor racemosus, Mycobacterium avium, Mycobacterium haemophilus, Mycobacterium leprae, Mycobacternon tuberculosis, Mycoplasma hominis, Mycoplasma ovis, Mycoplasma parvum, Mycoplasma pneumoniae, Neisserio elongare, Neisseria gonorrhoeae, Neisseria meningitidis, Nocardia nova, Paenibacillus lentus, Paraburkholderia fungorum, Pasteurella muitocida, Penicillium chrysogenum, Penicillium verrucosum, Pneumocystis carinii, Pneumocystis firovecii, Porphyromonas gingivalis, Prevotella dentalis, Prevotella denticola, Prevotella intermedia, Prevotella jejuni, Proteus mirabilis, Proteus vulgaris, Pseudoalteromonas marina, Pseudomonas aeruginosa, Pseudomonas fluorescens, Pseudomonas parafulva, Pseudomonas putida, Pseudomonas stutzeri, Ralstonia solanacearum, Rhodococcus fascians, Rickettsia japonica, Rickettsia prowazekii, Rickettsia rickettsia, Rothia dentocariosa, Saccharomyces cerevisiae, Salmonella bongori, Salmonella enterica, Scedosporium boydii, Schizosaccharomyces japonicus, Serratia liquefaciens, Serratia marcescens, Serratia quinivorans, Shewanella denitrificans, Shewanella halifaxensis, Shigella boydii, Shigella dysenteriae, Shigella flexneri, Shigella sonnei, Sphingomonas sp. AAP5, Spiroplasma citri, Spiroplasma gladiatoris, Spiroplasma kunkelii, Staphylococcus argenteus, Staphylococcus aureus, Staphylococcus auricularis, Staphylococcus capitis, Staphylococcus caprae, Staphylococcus epidermidis, Staphylococcus haemolyticus, Staphylococcus hominis, Staphylococcus kloosii, Staphylococcus lugdunensis, Staphylococcus lutrae, Staphylococcus schleiferi, Staphylococcus sciuri, Staphylococcus succinis, Staphylococcus warneri, Streptococcus agalactiae, Streptococcus australis, Streptococcus dysgalactiae, Streptococcus gordonii, Streptococcus lutetiensis, Streptococcus mitis, Streptococcus mutans, Streptococcus oralis, Streptococcus pasteurianus, Streptococcus pneumoniae, Streptococcus pyogenes, Streptococcus salivarius, Streptococcus sanguinis, Streptococcus urinalis, Streptococcus viridans, Treponema caldarium, Treponema pullidum, Ureaplasma urealyticum, Vibrio cholerae, Vibrio fischeri, Vibrio parahaemolyticus, Vibrio vulnificus, Xanthomonas campestris, Xanthomonas perforans, Yersinia enterocolitica, Yersinia pestis, Yersinia pseudotuberculosis, Yersinia ruckeri, Zobellella denitrificans, and Zymomonas mobilis. In an embodiment of the invention, the microbial agent is a bacterium belonging to a species selected from the group consisting of Acetoanaerobium sticklandii, Acholeplasma oculi, Achromobacter denitrificans, Acidimicrobium ferrooxidans, Acidiphilium multivorum, Acidovorax citrulli, Acinetobacter baumannii, Acinetobacter calcoaceticus, Acinetobacter guillouiae, Acinetobacter haemolyticus, Acinetobacter lactucae, Acinetobacter schindleri, Actinobacillus pleuropneumoniae, Actinobacillus succinogenes, Actinomyces slackil, Actinomyces viscosus, Aerococcus urinae, Aeromonas salmonicida, Aeromonas veronii, Anaplasma ovis, Anoxybacillus flavithermus, Bacillus anthracis, Bacillus licheriformis, Bacillus litoralis, Bacillus megaferium, Bacillus mycoides, Bacillus subtilis, Bacillus thuringiensis Bacteroides caccae, Bacteroides fragilis, Bacteroides thetaiotoomicron, Bacteroides vulgatus, Bartonella henselae, Bartonella quintana, Bartonella vinsonii, Bifidobacterium adolescentis, Bifidobacterium bifidum, Bifidobacterium gallinarum, Blottabacterium clevelandi, Bordetella bronchialis, Bordetella bronchiseptica, Bordetella parapertussis, Bordetella pertussis, Borrelia hermsii, Borreliella burgdorferi, Brachyspira murdochii, Brevibacterium aurantiacum, Burkholderia ambifaria, Burkholderia contaminans, Burkholderia mallei, Burkholderia pseudomallei, Burkholderia thailandensis, Campylobacter coli, Campylobacter jejuni, Campylobacter lari, Campylobacter ureolyticus, Capnocytophaga gingivalis, Caulobacter mirabilis, Chlamydia gallinacean, Chlamydia pneumoniae, Chlamydia psittaci, Chlamydia trachomatis, Citrobacter braakii, Citrobacter freundii, Citrobacter pasteurii, Citrobacter youngae, Clostridioides difficile. Clostridium acetobutylicum, Clostridium botulinum. Clostridium perfringens, Clostridium sporogenes, Corynebacterium diphtheriae, Corynebacterium flavescens, Corynebacterium glutamicum, Corynebacterium pseudotuberculosis, Corynebacterium simulans, Corynebacterium ulcerans, Coxiella burnetii, Dechloromonas aromatica, Desulfitobacterium hafniense, Edwardsiella tarda, Ehrlichia chaffeensis, Enterobacter bugandensis, Enterobacter cloacae, Enterobacter ludwigii, Enterococcus faecalis, Enterococcus faecium, Enterococcus gullinarum, Erwinia persicina, Escherichia coli, Escherichia fergusonii, Flavobacterium branchiophilum, Flavobacterium indicum, Flavobacterium johnsoniae, Flavobacterium pallidum, Francisella noutunensis, Francisella tularensis, Fusobacterium nucleatum, Fusobacterium ulcerans, Gordonia bronchiolis, Haemophilus influenzae, Haemophilus parainfluenzae, Helicobacter pylori, Klebsiella oxytoca, Klebsiella pneumoniae, Klebsiella variicola, Kluyvera intermedia, Lactobacillus acidophilus, Lactobacillus backit, Lactobacillus buchneri, Lactobacillus delbrueckii, Lactobacillus johnsonii, Lactobacillus rhamnosus, Lactococcus lactis, Legionella israelensis, Legionella pneumophila, Leptospira interrogans, Leptospira santarosai, Leuconostoc kimchi, Listeria innocua, Listeria monocytogenes, Microbacterium hominis, Moraxella catarrhalis, Moraxella cuniculi, Mycobacterium avium, Mycobacterium haemophilum, Mycobacterium leprae, Mycobacterium tuberculosis, Mycoplasma hominis, Mycoplasma ovis, Mycoplasma parvum, Mycoplasma pneumoniae, Neisseria elongate, Neisseria gonorrhoeae, Neisseria meningitidis, Nocardia nova, Paenibacillus lentus, Paraburkholderia fungorum, Pasteurella multocida, Porphyromonas gingivalis, Prevotella dentalis, Prevotella denticola, Prevotella intermedia, Prevotella jejuni, Proteus mirabilis, Proteus vulgaris, Pseudoalteramonas marina, Pseudomonas aeruginosa, Pseudomonas fluorescens, Pseudomonas parafulva, Pseudomonas putida, Pseudomonas stutzeri, Ralstonia solanacearum, Rhodococcus fascians, Rickettsia japonica, Rickettsia prowazekii, Rickettsia rickettsia, Rothia democariosa, Salmonella bongori, Salmonella enterica, Serratia liquefaciens, Serratia marcescens, Serratia quinivorans, Shewanella denitrificans, Shewanella halifaxensis, Shigella boydii, Shigella dysenteriae, Shigella flexneri, Shigella sonnei, Sphingomonas sp. AAP5, Spiroplasma citri. Spiroplasma gladiatoris, Spiroplasma kunkelii, Staphylococcus argenteus, Staphylococcus aureus, Staphylococcus auricularis, Staphylococcus capitis, Staphylococcus caprae, Staphylococcus epidermidis, Staphylococcus haemolyticus, Staphylococcus hominis. Staphylococcus kloosii, Staphylococcus lugdunensis, Staphylococcus lutrae, Staphylococcus schleiferi, Staphylococcus sciuri, Staphylococcus succinus, Staphylococcus warneri, Streptococcus agalactiae, Streptococcus australis, Streptococcus dysgalactiae, Streptococcus gordonii, Streptococcus lutetiensis, Streptococcus mitis, Streptococcus mutans, Streptococcus oralis, Streptococcus pasteurianus, Streptococcus pneumoniae, Streptococcus pyogenes. Streptococcus salivarius, Streptococcus sanguinis, Streptococcus urinalis, Streptococcus viridans, Treponema caldarium, Treponema pallidum, Ureaplasma urealyticum, Vibrio cholerae, Vibrio fischeri, Vibrio parahaemolyticus, Vibrio vulnificus, Xanthomonas campestris, Xanthomonas perforans, Yersinia enterocolitica, Yersinia pestis, Yersinia pseudotuberculosis, Yersinia ruckeri, Zobellella denitrificans, and Zymomonas mobilis. In an embodiment of the invention, the microbial agent is a fungus belonging to a species selected from the group consisting of Aspergillus clavatus, Aspergillus fumigatus, Aspergillus niger, Aspergillus oryzae, Aspergillus terreus, Candida albicans, Candida auris, Candida blattae, Candida castellii, Candida dubliniensis, Candida glabrata, Candida intermedia, Candida metapsilosis, Candida orthopsilosis, Candida parapsilosis, Candida rhagii, Candida sake, Candida tropicalis, Cunninghamella bertholletiae, Fusarium oxysporum, Fusarium secorum, Fusarium verrucosum, Histoplasma capsulatum, Kluyveromyces lactis, Kluyveromyces nonfermentans, Malassezia dermatis, Malassezia furfur, Molossezio pachydermatis, Mucor racemosus, Penicillium chrysogemim, Penicillium verrucosum, Pneumocystis carinii, Pneumocystis jirovecii, Saccharomyces cerevisiae, Seedosporium boydii, and Schizosaccharomyces japonicus.

In an embodiment of the invention, the Enterococcus faecium comprises vanA gene.

In an embodiment of the invention, the Staphylococcus aureus comprises a mecA gene.

In an embodiment of the invention, the Klebsiella pneumoniae comprises a KPC gene.

In an embodiment of the invention, the sample may comprise more than one microbial agent provided in but not limited to those in Table 7. In an embodiment of the invention, the microbial agents may be any of, but not limited to, genus or species as listed in Table 7. In an embodiment of the invention, the microbial agents may be a combination of microbial agents provided by not limited to those in Table 7. In a further embodiment, the microbial agent may comprise a drug resistant marker or gene.

In an embodiment of the invention, the subject is a sepsis candidate, has sepsis, or is otherwise suspected of having a bloodstream infection.

In an embodiment of the invention, the subject is infected with a common-sepsis-associated yeast Candida albicans.

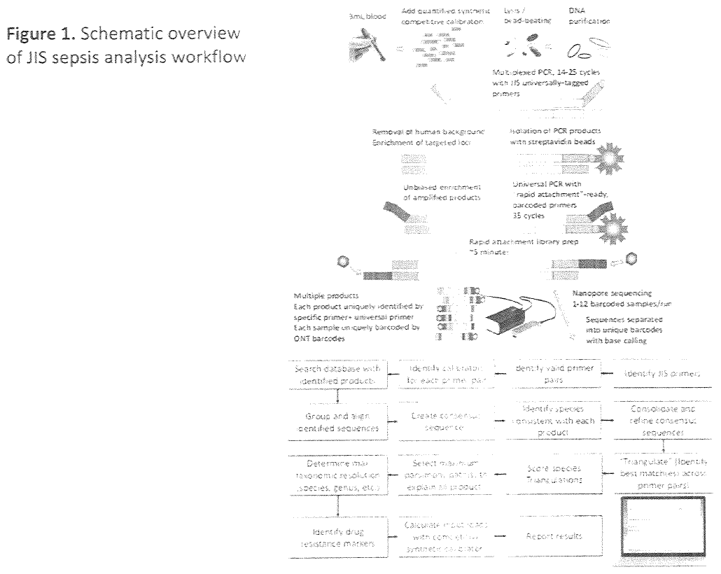

In an embodiment of the invention, the method for detecting the presence, identifying, and/or quantifying the amount of an unidentified microbial agent in a sample comprises a workflow as shown in FIG. 1.

In an embodiment of the invention, the method for detecting the presence, identifying, and/or quantifying the amount of an unidentified microbial agent in a sample comprises a workflow as shown in FIG. 1.

The invention provides a method for enhancing gravity sedimentation of red blood cells in a blood sample comprising:
a) contacting the blood sample with a starch so that red blood cells shrink in volume and aggregate; and
b) permitting the red blood cell aggregate to settle to bottom of a vessel, thereby enhancing gravity sedimentation of the red blood cells in a blood sample.

In an embodiment of the invention, the starch is hetastarch or hydroxy-ethyl starch (HES).

For example, the blood sample may be about 3 mL. In an embodiment of the invention, the red blood cell aggregate sediments within about 15 min, leaving a red blood cell-depleted blood fraction of about one-half original volume or about 1.5 mL. In an embodiment of the invention, sedimentation of the red blood cell occurs in the absence of centrifugation. In an embodiment of the invention, the blood sample is a whole blood sample.

Additionally, the invention provides a method for depleting a blood sample of red blood cells comprising:
a) contacting the blood sample with a starch so that red blood cells shrink in volume and aggregate; and
b) permitting the red blood cell aggregate to settle to bottom of a vessel, thereby depleting a blood sample of red blood cells.

Merely by way of example, the starch may be a hetastarch or hydroxy-ethyl starch (HE'S). The blood sample may be about 3 mL. In an embodiment of the invention, the red blood cell aggregate sediments within about 15 min, leaving a red blood cell-depleted blood fraction of about one-half original volume or about 1.5 mL. In an embodiment of the invention, sedimentation of the red blood cell is by gravity sedimentation. In an embodiment of the invention, sedimentation of the red blood cell occurs in the absence of centrifugation. In an embodiment of the invention, the blood sample is cleared or depleted of red blood cells in absence of centrifugation. The blood sample may be a whole blood sample.

The invention further provides a method for removing an enzymatic inhibitor associated with red blood cells for a downstream procedure requiring lysis of cells in a blood sample comprising:
a) contacting the blood sample with a starch so that red blood cells shrink in volume and aggregate;
b) permitting the red blood cell aggregate to settle to bottom of a vessel; and
c) isolating remaining blood volume depleted of red blood cells, thereby removing enzymatic inhibitors associated with red blood cells for a downstream procedure requiring lysis of cells in a blood sample.

In an embodiment of the invention, the enzymatic inhibitor is hemoglobin or heme.

In an embodiment of the invention, the enzymatic inhibitor inhibits DNA polymerase.

In an embodiment of the invention, the DNA polymerase is used in a polymerase chain reaction.

In an embodiment of the invention, the starch is hetastarch or hydroxy-ethyl starch (HES). The blood sample may be about 3 mL. The red blood cell may aggregate sediments within about 15 min, leaving a red blood cell-depleted blood fraction of about one-half original volume or about 1.5 mL. In an embodiment of the invention, the method is free of centrifugation. The blood sample may be a whole blood sample.

Further provides is a method for separating a microbial agent from red blood cells in a blood sample comprising:
a) contacting the blood sample with a starch so that red blood cells shrink in volume and aggregate;
b) permitting the red blood cell aggregate to settle to bottom of a vessel; and
c) removing remaining blood volume depleted of red blood cells, thereby separating a microbial agent from red blood cells in a blood sample.

In an embodiment of the invention, the starch is hetastarch or hydroxy-ethyl starch (HES). The blood sample may be about 3 mL. The red blood cell may aggregate sediments within about 15 min, leaving a red blood cell-depleted blood fraction of about one-half original volume or about 1.5 mL. The method may be free of centrifugation. The blood sample may be a whole blood sample.

Also provided by the invention is a method for lysing bacteria and/or fungi in a liquid sample comprising:
a) introducing the bacteria and/or fungi suspension to an agitator comprising a chamber, directional blades, disrupting beads and a motor;
b) operating the agitator in a cycling routine of short pulses of alternating direction so as to fracture bacteria and/or fungi releasing nucleic acid, thereby lysing bacteria and/or fungi in the liquid sample.

In an embodiment of the invention, the blades are alternating directional blades.

In an embodiment of the invention, the beads are yttrium-zirconium beads.

In an embodiment of the invention, each pulse is about 2 seconds.

In an embodiment of the invention, the bacteria and/or fungi are stained Gram-positive or Gram-negative.

The method of the invention may be capable of lysing difficult to lyse Gram-staining bacterium or fungus. By way of example, the Gram-staining bacterium may be Staphylococcus spp., Enterococcus spp., Streptococcus spp., Bacillus spp., Clostridium spp. or any other of a large variety of Gram(+) bacteria. By way of example, the Gram-staining fungus may be Cryptococcus spp., Candida spp., Aspergillus spp., Saccharomyces spp., Scedosporium spp. or any other of a large number of fungi and yeast.

Also, the invention provides a method of treating a subject infected with an unidentified microbial agent comprising:
i) obtaining a sample from the subject;
ii) determining the presence of the unidentified microbial agent and determining its identity by the method of the invention;
iii) administering an effective appropriate anti-microbial agent so as to reduce or eliminate the infection.

The invention also provides a method for lysing bacteria and/or fungi in a liquid sample comprising:
i) introducing the bacteria and/or fungi suspension to a cell lysis device;
ii) operating the device in a cycling routine of short pulses of alternating direction so as to fracture bacteria and/or fungi releasing nucleic acid, thereby lysing bacteria and/or fungi in the liquid sample.

The invention provides a kit comprising any one of the primers or primer pairs as provided in Tables 2 and 5, any of the calibrator molecules as provided in Table 6, and instructions. In an embodiment, a kit of the invention may comprise one or more primer or primer pair directed to nucleic acid sequences specified by Coordinates of Reference Sequences provided in Table 1 and 4. The kit may have one or more primer or primer pair directed a Reference Sequence of Table 1 or 4, or a combination of primers or primer pairs for a combination of Reference Sequences of Table 1 and 4. The kit may additionally include instruction for use with the device of the invention. The kit may accompany the cell lysis device of the invention. The kit may accompany the system of the invention.

Advantages of the invention includes an end-to-end integration of the following strategies: a. Increased sensitivity for multiplexed analysis through pre-removal of amplification inhibitors from blood; b. Compact energy- and space-efficient cellular disruption and DNA shearing through the use of a rapidly-rotated, alternating direction paddle combined with yttrium-zirconium beads; c. Increased sensitivity through multiplexing of broadly-targeted primers to allow concentration of extracted nucleic acids into one reaction; d. Minimization of amplification bias in the multiplexed PCR by a two-stage process with targeted amplification only proceeding for a few cycles flowed by universal amplification with one primer pair; e. Removal of human DNA background using tagged primers prior to universal amplification; f. Increased accuracy of nanopore sequence outputs through consensus analysis; g. Nearly agnostic identification of blood-borne pathogens through universal and broad amplification targeting of bacteria and fungi; h. Increased breadth of coverage for novel pathogens compared to competition; and i. Quantification of pathogen load in the original sample through comparison of pathogen amplification to the output of competitive calibrator molecule amplification.

### Devices of the Invention

Additionally, the invention provides devices for cell lysis and systems for detecting the presence, identifying, and/or quantifying amount of an unidentified microbial agent in a sample, where the sample may be from a subject, such as a human.

In one embodiment, the device for cell lysis comprises an open chamber comprising a closed wall and an open end, a cap holder for an alternating directional blade and to close the open end of the chamber, an alternating directional blade, a motor to attach and rotate to the blade, and a controller to control speed and direction of rotation of the blade.

In another embodiment, the device for cell lysis comprises an open chamber comprising a closed wall and an open end, a cap holder for an alternating directional blade and to close the open end of the chamber, an alternating directional blade, a motor to attach and rotate to the blade, a controller to control speed and direction of rotation of the blade and a power source.

In a separate embodiment, the device for cell lysis comprises an open chamber comprising a closes wall and an open end, a cap holder for an alternating directional blade and to close the open end of the chamber, an alternating directional blade, a motor to attach and rotate to the blade, a controller to control speed, direction of rotation of the blade and a power source and disrupting beads. In an embodiment, the disrupting beads are yttrium-zirconium beads

In an embodiment, the chamber of the device for cell lysis is a cylinder. The wall of the chamber may be made of plastic or glass. The chamber may comprise one closed end and one open end, or alternatively, two open ends which can be closed on one end with the cap holder and the other with a lid or a plug. The one closed end of the chamber may be conical or rounded. In an embodiment, the cap holder attaches to the blade and attaches to the open end of the chamber. In an embodiment, the chamber when fully assembled and operating is a closed chamber.

In an embodiment, the fully assembled chamber comprises space to accommodate a liquid sample, disrupting heads, the blade and optionally an air space between the cap holder and top of the liquid sample.

In an embodiment, clearance of the blade from the chamber wall is about 1-5 mm in its central and lower portion.

In an embodiment, the blade region spans 50-90 percent of the vertical height of the fully assembled chamber space.

In an embodiment, the blade comprises rectangular solid blocks or cubes protruding from a central axis. In an embodiment, the blocks and/or cubes comprise alternating orientations along the central axis of the blade, such that no two adjacent blocks and/or cubes are oriented in the same direction. The blade can be tapered at its tip relative to its main body.

In an embodiment, the blade attached to the cap is as shown in FIG. 2 and on an assembled solid support is as shown in FIG. 10A. In an embodiment, the blade has the specification with dimensions or relative dimension and orientation of rectangular solid blocks or cubes of the blade as shown in FIG. 12D. In an embodiment, the blade is inserted into a tube whose bottom is conical or round. In a preferred embodiment, the tube bottom or chamber bottom is conical. The tip of the blade can be tapered. In an embodiment, the tip of the can be tapered and comprise ridges. The blade can be sterile. The blade can be free of contaminating microbial agents prior to use. In an embodiment, the blade is sterile or free of contaminating microbial agents prior to use. In an embodiment, interior surfaces of the chamber is sterile or free of contaminating microbial agents prior to use.

In an embodiment, the fully assembled chamber can hold a volume of liquid between 1-3 mL. In an embodiment, the fully assembled chamber can hold a volume of liquid between 1-3 mL and also air space between the cap holder and top of the liquid sample. In an embodiment, the fully assembled chamber is filled with a liquid sample between 1-3 ml, wherein height of the liquid sample is at or below top of last protuberance from spindle or axis of the blade closest to the cap holder.

In an embodiment, the blade may be rotated in a clockwise or counterclockwise direction. In an embodiment, the motor rotates the blade clockwise or counter-clockwise. In an embodiment. In an embodiment, the blade may be rotated in a clockwise or counterclockwise direction powered by a motor.

In an embodiment, the motor is equipped with a toothed gear that meshes to a complementary gear acceptor attached to the blade through a sealed bearing in the cap for the chamber. In an embodiment, the motor produces a cycling routine of short pulses of alternating direction. Each pulse can be about 2 seconds.

In an embodiment, the motor operates with a 12-15V power supply. In an embodiment, the motor is powered by direct current. In an embodiment, the motor is powered by a power supply producing direct current. In an embodiment the motor is attached to a controller. In an embodiment, the controller controls the rotation of the motor. In an embodiment, the controller controls the motor so as produce cycles of short pulses in alternating direction. In an embodiment, the controller is attached to the motor to produce cycles of short pulses in alternating direction. In an embodiment, the power source is attached to a controller which in turn is attached to the motor to produce cycles of short pulses in alternating direction. In an embodiment, each pulse is about 2 seconds.

In an embodiment, the motor is attached to a controller. In an embodiment, the controller is a microcontroller. By way of example, the microcontroller can be an Arduino Nano microcontroller or equivalent. In an embodiment, the microcontroller comprises an automated run mode to control the motor. In an embodiment, the microcontroller can be controlled by serial connection to a PC-based controller application. In an embodiment, the PC-based controlled application is written in C#.NET. In an embodiment, the PC-based controlled application is written for Windows OS.

In an embodiment, the toothed gear comprises a concentric ring of v-shaped teeth on a cylindrical rod or spindle. In an embodiment, the toothed gear comprises anywhere from 8 to 16 teeth. In a preferred embodiment, the toothed gear comprises 12 teeth as shown in FIG. 10D top panel. In a preferred embodiment, the toothed gear comprising 12 teeth has a specification with dimensions or relative dimensions as shown in FIG. 12G.

In an embodiment, the complementary gear acceptor attached to the blade through a sealed bearing in the cap for the chamber meshes with the toothed gear. In an embodiment, the complementary gear acceptor meshes with a toothed gear chosen from a range of 8-16 teeth. In a preferred embodiment, the complementary gear acceptor meshes with a 12-teeth gear as shown in FIG. 12E. In a preferred embodiment, the complementary gear acceptor meshing with 12-teeth gear has a specification with dimensions or relative dimensions as shown in FIG. 12E.

In an embodiment, the motor is attached to a solid support which provides band-free disruption of a microbial agent. The microbial agent may comprise a cell wall and not be easily disruptable. In an embodiment, the motor is one of two or more motors attached to a solid support which provides hand free disruption of the microbial agent and optionally multiple sample disruptions simultaneously. In an embodiment, the solid support comprises a spring loaded housing fixed to each motor, wherein the spring-loaded housing permit convenient securing and meshing of the motor to the complementary gear acceptor attached to the cap used to seal the chamber. The sealed chamber can be a chamber of a plastic or glass tube. In an embodiment, the solid support further comprises port for motor wiring, slide-in locking latch, vertical tube support, a base stand, and a top support for motor mount.

In an embodiment, the solid support allows simultaneous processing of samples in three separate chambers. In a preferred embodiment, the solid support fully or partially assembled with or without the chambers is as shown in FIGS. 9 to 12. In a preferred embodiment, the solid support has a specification with dimensions or relative dimensions as shown in FIGS. 9 to 12.

In an embodiment, the device for cell lysis is presented in FIG. 2.

In a preferred embodiment, the device is presented in FIG. 9.

In an embodiment, the device can be a 3D-printed device, as presented in FIG. 10.

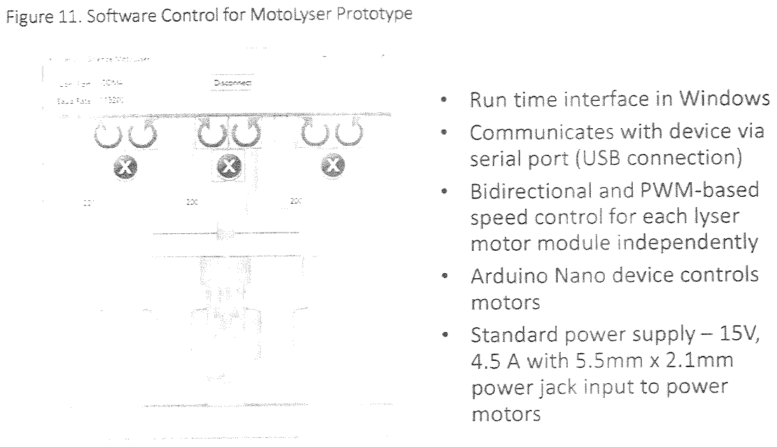

In an embodiment, the device can be controlled by a microcontroller, in turn controlled by a PC application through a serial USB connection, as presented in FIG. 11.

In an embodiment, the 3D printed designs of device parts may be those presented in FIG. 12.

In one embodiment, the system is for detecting the presence, identifying, and/or quantifying an amount of an unidentified microbial agent in a sample. In an embodiment, the system performs the steps of:
a) Mixing a sample with quantified calibrator molecules;
b) Isolating nucleic acid in the sample;
c) Interrogating and amplifying select nucleic acid sequences with a collection of primer pairs so as to produce double-stranded DNAs, wherein each double-stranded DNA comprises an identifier nucleic acid sequence, a universal amplification target sequence, and a chemical tag;
d) Isolating the double-stranded DNAs so produced in step (c) using the chemical tag;
e) Determining DNA sequence of the isolated double-stranded DNAs;
f) Identifying and quantifying the output of the calibrator sequences added in step (a)
g) Organizing DNA sequences so obtained in step (e) to produce a collection of consensus sequences; and
h) Comparing the collection of consensus sequences against reference sequences so as to detect the presence of an unidentified microbial agent and identify the microbial agent in the sample; and
i) Optionally, comparing the signal output levels from each agent identified in the sample to the output levels of the calibrator molecules in order to quantify the genome copy amount of the agent in the original sample.

In an embodiment, the system performs the steps of:
a2) Adding quantitative calibrator molecules to the sample;
b2) Isolating nucleic acid in the sample;
c2) Interrogating and amplifying select nucleic acid sequences with a collection of primer pairs so as to produce double-stranded DNAs, wherein each double-stranded DNA comprises an identifier nucleic acid sequence, a chemical tag, and optionally a DNA barcode label;
d2) Isolating the double-stranded DNAs so produced in step (c2) using the chemical tag;
e2) Determining DNA sequence of the isolated double-stranded DNAs;
f2) Identifying and quantifying the output of the calibrator sequences added in step (a2);
g2) Organizing DNA sequences so obtained in step (e) to produce a collection of consensus sequences;
h2) Comparing the collection of consensus sequences against reference sequences so as to detect the presence of an unidentified microbial agent and identify the microbial agent in the sample; and
i2) Optionally, comparing the signal output levels from each agent identified in the sample to the output levels of synthetic calibrators in order to quantify the genome copy amount of the agent in the original sample.

In an embodiment, the system performs the steps of:
b) Isolating nucleic acid in the sample;
c) Interrogating and amplifying select nucleic acid sequences with a collection of primer pairs so as to produce double-stranded DNAs, wherein each double-stranded DNA comprises an identifier nucleic acid sequence, a universal amplification target sequence, and a chemical tag;
d) Isolating the double-stranded DNAs so produced in step (c) using the chemical tag;
e) Determining DNA sequence of the isolated double-stranded DNAs;
f) Identifying and quantifying the output of the calibrator sequences added in step (a)
g) Organizing DNA sequences so obtained in step (e) to produce a collection of consensus sequences; and
h) Comparing the collection of consensus sequences against reference sequences so as to detect the presence of an unidentified microbial agent and identify the microbial agent in the sample; and
i) Optionally, comparing the signal output levels from each agent identified in the sample to the output levels of the calibrator molecules in order to quantify the genome copy amount of the agent in the original sample.

In an embodiment, the system performs the steps of:
b2) Isolating nucleic acid in the sample;
c2) Interrogating and amplifying select nucleic acid sequences with a collection of primer pairs so as to produce double-stranded DNAs, wherein each double-stranded DNA comprises an identifier nucleic acid sequence, a chemical tag, and optionally a DNA barcode label;
d2) Isolating the double-stranded DNAs so produced in step (c2) using the chemical tag;
e2) Determining DNA sequence of the isolated double-stranded DNAs;
f2) Identifying and quantifying the output of the calibrator sequences added in step (a2);
g2) Organizing DNA sequences so obtained in step (e) to produce a collection of consensus sequences;
h2) Comparing the collection of consensus sequences against reference sequences so as to detect the presence of an unidentified microbial agent and identify the microbial agent in the sample; and
i2) Optionally, comparing the signal output levels from each agent identified in the sample to the output levels of synthetic calibrators in order to quantify the genome copy amount of the agent in the original sample.

In an embodiment, the system additionally performs the steps of:
i) separating the unidentified microbial agent from the sample, and
ii) disrupting the separated microbial agent so that the agent fractures, exposes and releases its nucleic acid prior to isolating nucleic acid in the sample.

In an embodiment, the system is an integrated system where all steps performed are integrated in the system.

In an embodiment, the system is automated or semi-automated. In an embodiment, the system is automated so as to not require a user to intervene once the system is setup and Started. In an embodiment, the system is semi-automated requiring user intervention for at least one step but not all steps once the system is setup and started.

In an embodiment, the system is a point-of-care system. In an embodiment, the point-of-care is a physician office or a clinic. In an embodiment, the point-of-care is a hospital. In an embodiment, the system is used in a laboratory or research setting. In an embodiment, the system is used in a remote setting with minimal infrastructural support or rural setting.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

### Example 1

The current embodiment targets 3 ml of human blood to balance a relatively small volume requirement with the need to ensure sufficient material to detect organisms that may be present at levels as low as 1-10 cfu/mL. An automation-friendly extraction procedure that uses aqueous buffers and can be performed without large equipment or centrifugation have been developed. The extraction procedure utilizes hetastarch to condense red blood cells, which can passively reduce the cleared blood fraction to ˜½ the original volume by gravity in ˜15 minutes (centrifugation can be used to speed the process), followed by yttrium-zirconium bead lysis in a compact motorized lysis device of our own design (˜5 minutes, FIGS. 2, 9, 10, 11 and 12).

The lysis device shown in FIGS. 2, 9 and 10 utilizes inexpensive 12V motors (˜14,000 rpm), an agitator with alternating directional blades, yttrium-zirconium beads, and a cycling routine of short (2 second) pulses of alternating direction to induce maximal frictional stress without the bulk and violence of a standard bead-beating instrument, making it compatible with incorporation into small instrumentation in an automated system.

It has been demonstrated that this device performs equivalently to a Bertin Minilyser bead-beater in the same time in side-by-side comparisons using split-volume hetastarch-cleared blood samples containing difficult to lyse organisms such as Staphylococcus aureus and Cryptococcus neoformans. After lysis, the sample prep workflow utilizes a pH-modulation chemical approach (e.g., ChargeSwitch (ThermoFisher)) that requires about IS minutes operation time and utilizes only three additional aqueous buffers and is compatible with automation. Alternatively, standard blood extraction methods such as Qiagen kits can also be used to produce nucleic acid compatible with our multiplexed assay system.

Our current molecular assay employs 50 primers in a single multiplex, allowing maximal concentration of extracted DNA into a single reaction (Table 4, and Table 5).

Primers have been designed to target maximally-conserved regions of bacterial or yeast/fungal DNA that surround systematically varying sequence regions while having minimal potential for interacting with human DNA or each other. As show in FIG. 1, each primer is also tagged at the 5′ end with universal target sequences compatible with the Oxford Nanopore Technologies (ONT) rapid-attachment and barcoding library preparation system. The combination of primer specificity region and universal target sequence is used in the downstream computational process to rapidly identify sequences generated from proper pairing primers to quickly weed out cross-reactive products which are from the magnitude of background DNA present in a human blood sample. One primer is 5′-labeled with biotin.

After addition of quantitative calibrators, and after RBC separation, lysis and DNA extraction (˜45 minutes), a single PCR reaction consisting of 30 uL extraction eluate and 20 uL. PCR master mix is thermocycled for 10-25 cycles (˜30-45 minutes) to create a seed population of markers maintained in the exponential phase of the amplification process (to minimize primer efficiency biases). This seed reaction utilizes a “touch-down-then-up” annealing strategy where initial primer annealing is performed at a high temperature, then drops below the calculated Tm's to allow annealing over target mismatches to a limited extent, increasing breadth of coverage, and then increasing annealing temperatures back up incrementally. Because the reaction happens in a heavy human DNA background, primer sequence design must be carefully balanced against mismatch tolerance to favor pathogen target amplification over human background amplification.

A high (500-2500 mM) concentration of betaine is utilized in the initial PCR reaction to help normalize hydrogen-bonding affinities between A. T and G-C base pairs for the initial seed PCR reaction. Amplified products are subjected to an exonuclease I digestion to fragment unused single-stranded primers and then removed from background DNA with streptavidin-coated paramagnetic beads, producing a low concentration of highly-enriched and universally-tagged target products. A PCR master mix containing a single pair of barcoded ONT rapid-attachment chemistry-equipped primers is added to the beads (after discarding the original PCR reaction) and thermocycled for 20-35 cycles (˜1 hour, beads do not need to be removed before PCR).

After PCR, an AMPure-based cleanup step is performed, a 5-minute, single-tube rapid-attachment library prep is performed, and the reaction is loaded into a flowcell in an ONT MinION USB sequencer. Sequencing is performed for 30 minutes to six hours and data are analyzed by Janus-I Science (JIS) proprietary software (NanoSepsID) (e.g., on a laptop computer), generally in 5-30 minutes, depending on the complexity of the sample.

Nanopore sequencing may be initiated and monitored by JIS software, may be performed on a scalable bank of NVIDIA GPU-equipped computational devices to utilize real-time GPU-based base-calling of sequence data, and data analysis may be automatically performed during sequencing and/or after sequence acquisition.

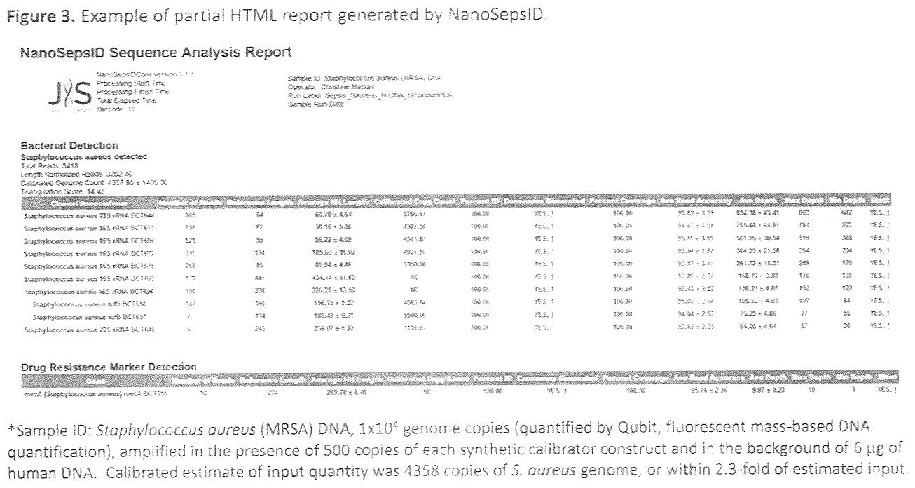

FIG. 3 shows the top section of a report for a sample containing MRSA (methicillin-resistant Staphylococcus aureus) DNA combined with 6 μg of human DNA. In this sample. 1×10⁴ copies of genomic DNA purified from Staphylococcus aureus carrying the mecA gene was added to 6 μg of DNA isolated from human blood with the addition of 500 copies of each synthetic calibrator molecule, subjected to the JIS multiplexed assay and six hours of nanopore sequence acquisition, followed by automated sample analysis. The quantitative readout estimated input DNA levels to within 2.3-fold of estimated inputs.

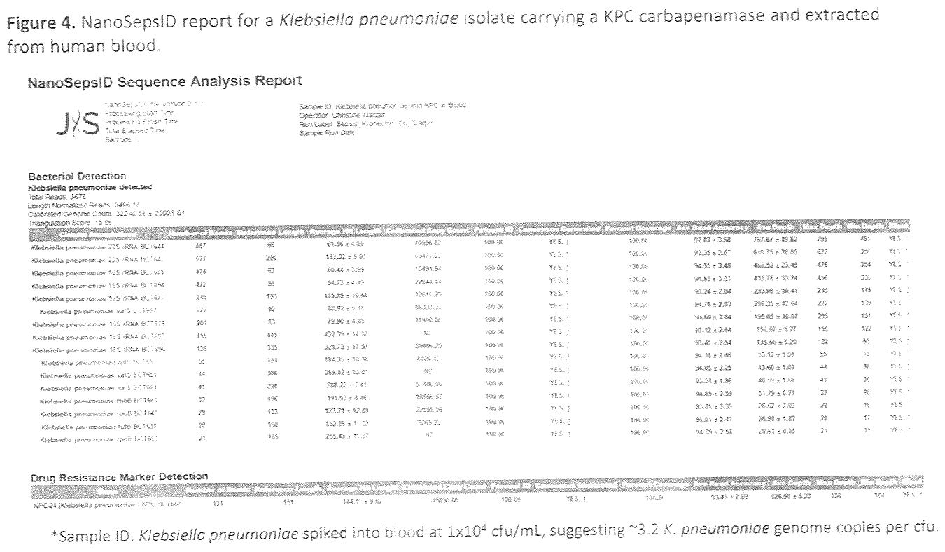

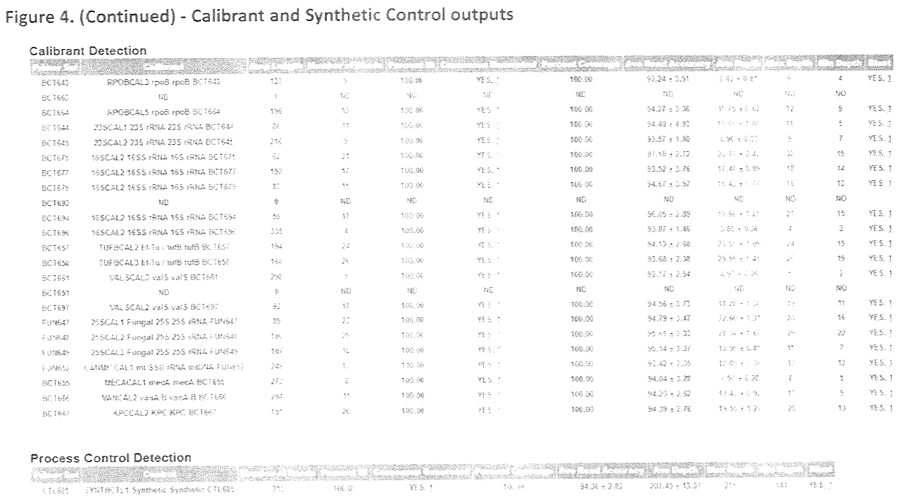

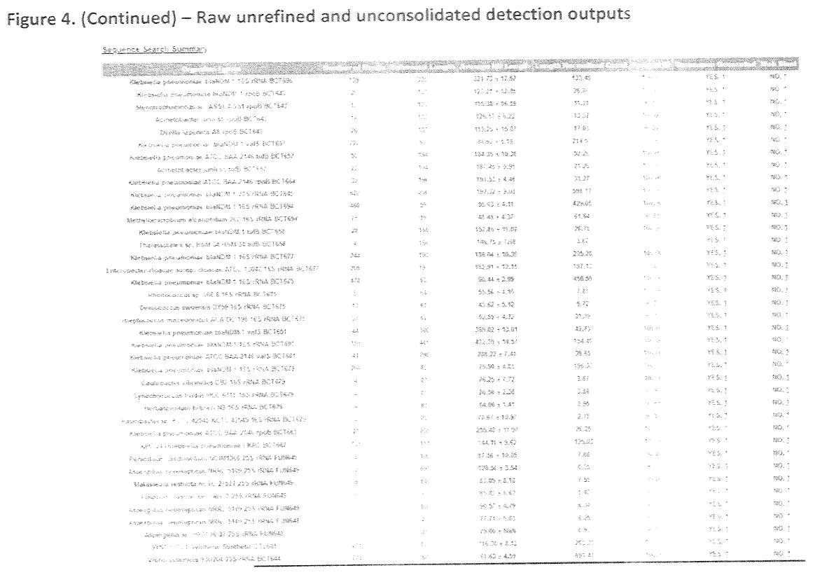

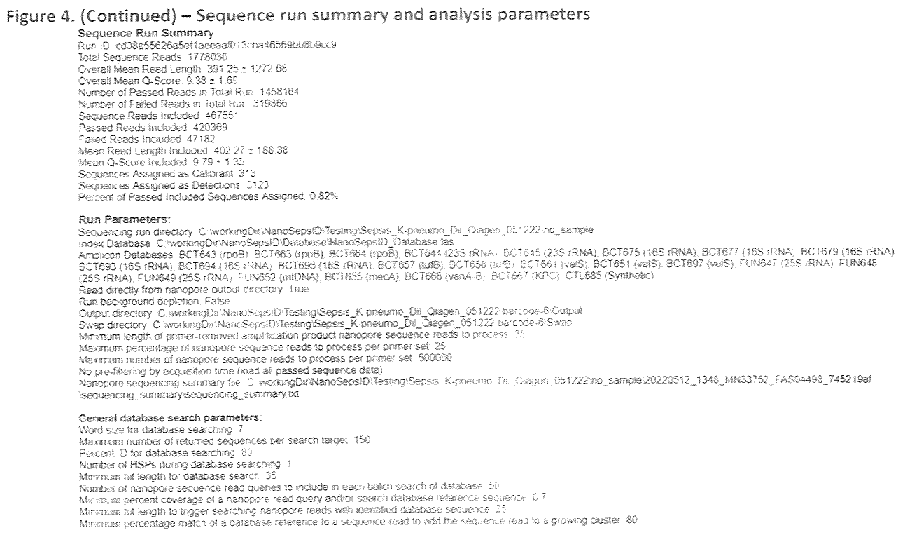

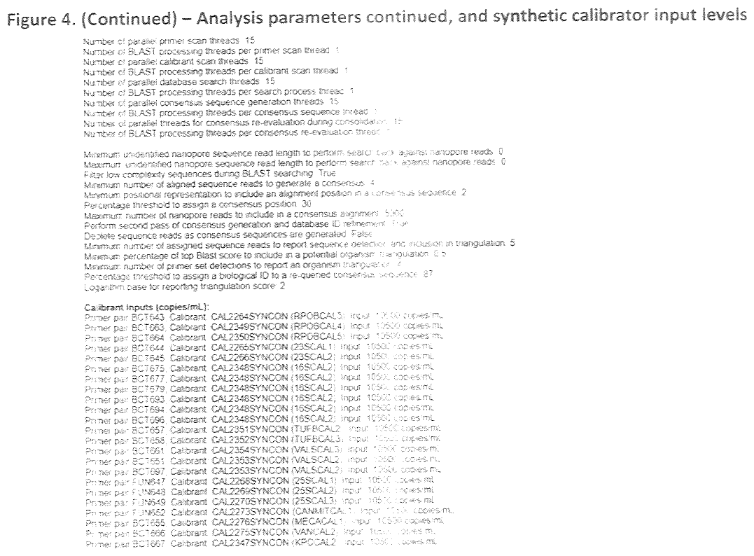

FIG. 4 shows a full report generated from a sample of Klebsiella pneumoniae (with KPC carbapenemase) extracted from human blood with a standard Qiagen blood extraction kit. This example, where 1×10⁴ cfu/ml, were added to the blood, suggests that ˜3.2 genome copies/cfa were effectively accessible in the sample, as indicated by the addition and co-extraction of 1.05×10⁴ calibrant copies per primer set into the blood sample prior to extraction. This report shows the full listing of detections as well as a summary of the sequencing run and the analysis parameters used in the analysis.

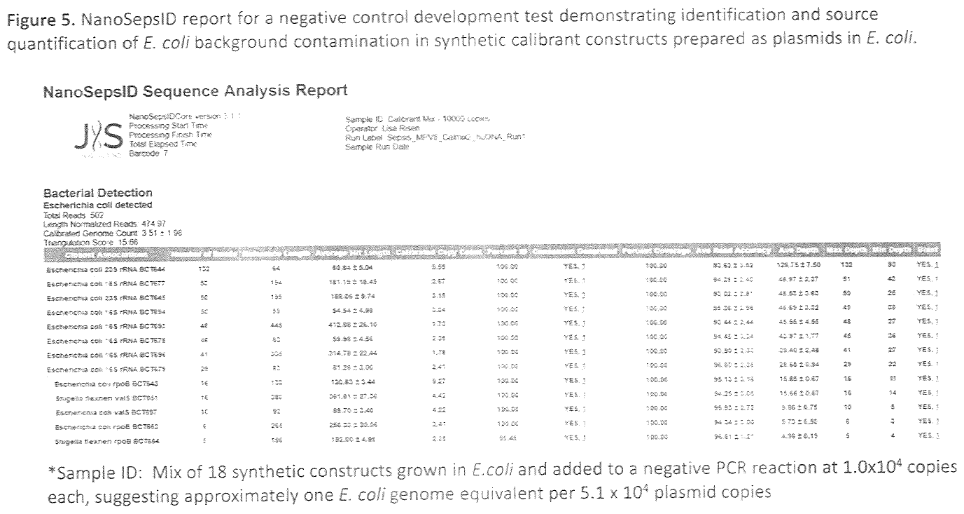

FIG. 5 shows a report generated from a negative sample testing only plasmid calibrator constructs added to the initial PCR reaction. The plasmid constructs were propagated in E. coli, which is detected in the background at a low level with an estimated quantity of ˜1 E. coli genome equivalent per 51,000 plasmid construct copies.

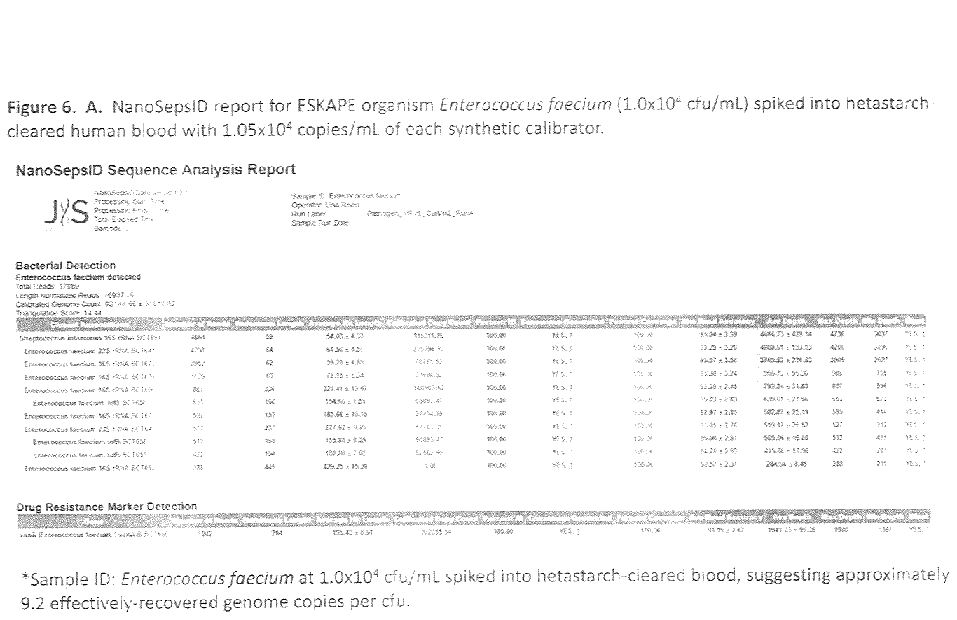

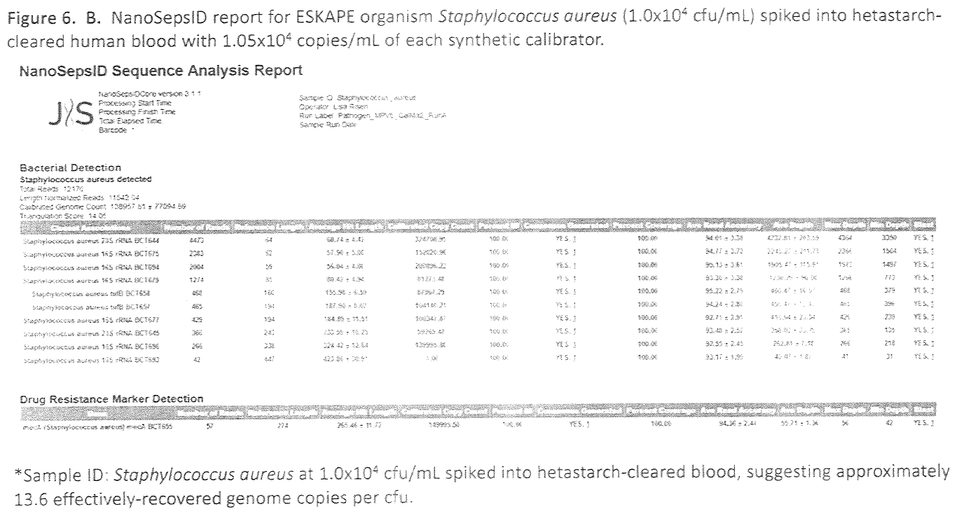

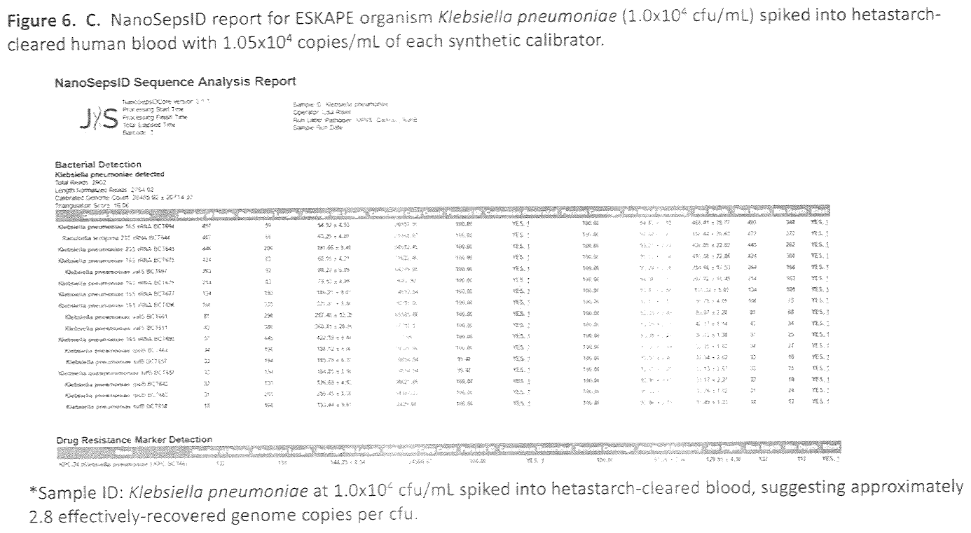

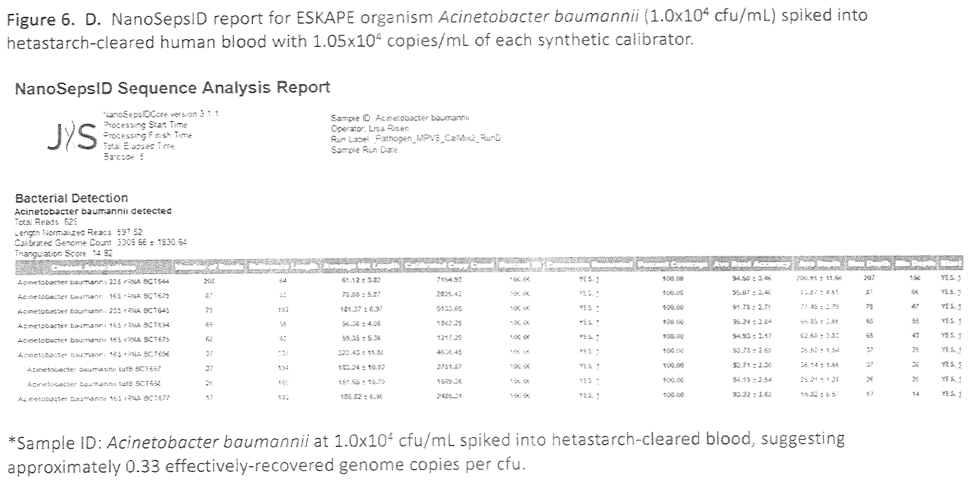

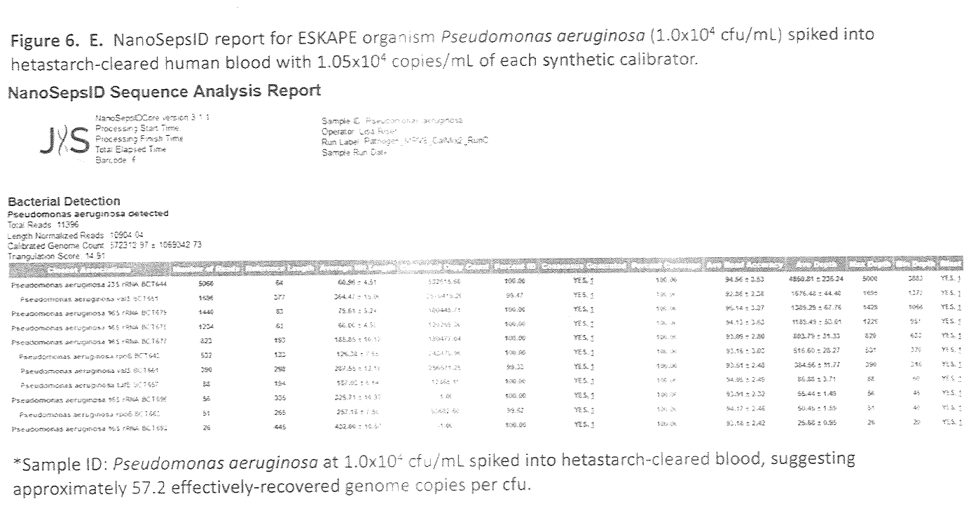

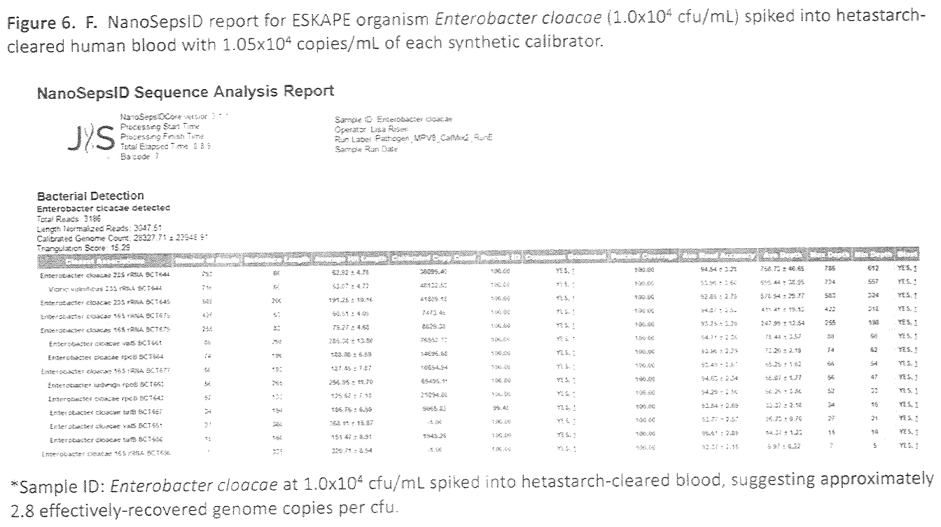

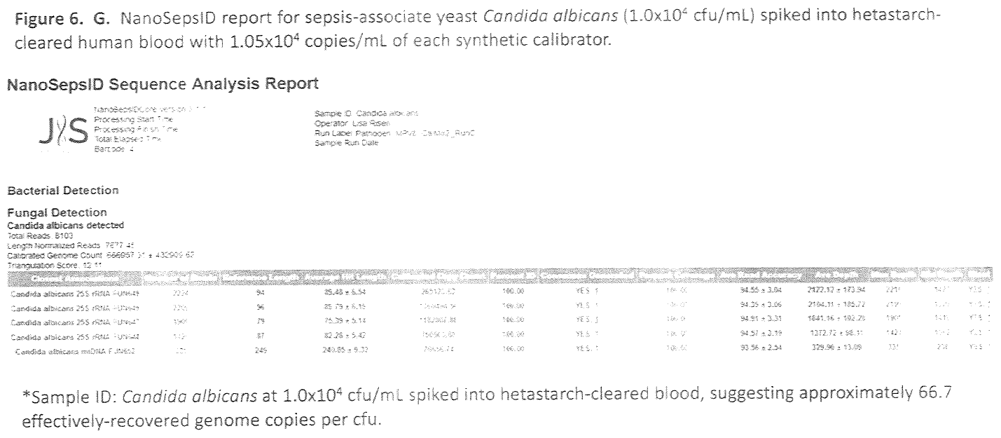

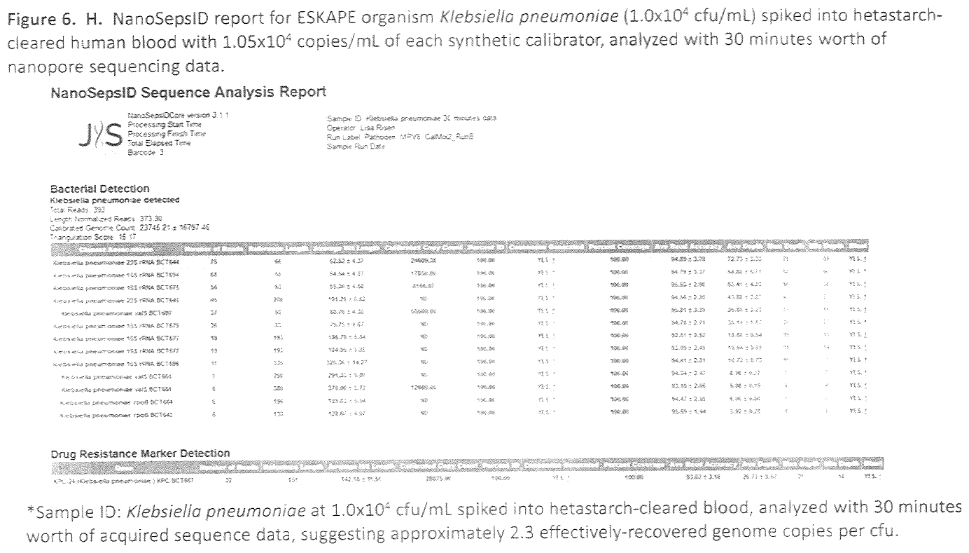

FIG. 6 shows an analysis of a representative of each of the ESKAPE organism categories, plus the common sepsis-associated yeast Candida albicans. Representative analyses are A. Enterococcus faecium carrying the vanA gene, suggesting-9.2 genome copies per cfu; B. Staphylococcus aureus carrying the mecA gene, suggesting ˜13.6 genome copies per cfu; C. Klebsiella pneumoniae carrying the KPC gene, suggesting ˜2.8 genome copies per cfu; D. Acinetobacter baumannii, suggesting ˜0.33 genome equivalents per cfu were effectively recovered; E. Pseudomonas aeruginosa, suggesting ˜57.2 genome copies per cfu; F. Enterobacter cloacae, suggesting ˜2.8 genome copies per cfu; and G. Candida albicans, suggesting-66.7 genome copies per cfu. As each of these were analyzed using six hours' worth of sequencing data (although run with two samples per flow cell simultaneously, so this is roughly equivalent to three hours of sequencing data for each sample), FIG. 6. H, shows a report for the Klebsiella pneumoniae sample analyzed using the first 30 minutes worth of sequencing data (equivalent to 15 minutes of sequencing for one sample on the flow cell). Despite several calibrant molecules not providing sufficient data for quantification, quantification is still automatically performed using the available calibrator molecules, and quantification of sample load is not substantially affected using the first 1/12 of the acquired sequence data (estimate of ˜2.3 genomes per cfu vs ˜2.8 genomes per cfu).

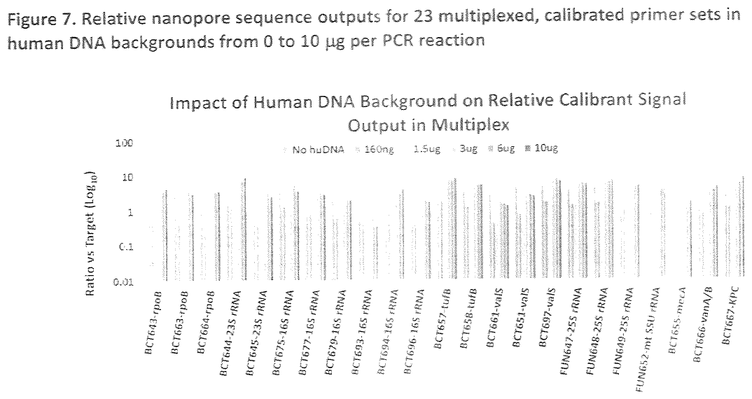

FIG. 7 shows the relative output of calibrant molecule signals (as assessed by number of amplicon sequences observed for each calibrator) in negative samples in a background of 0 to 10 μg of human DNA, demonstrating that overall output balance is largely maintained.

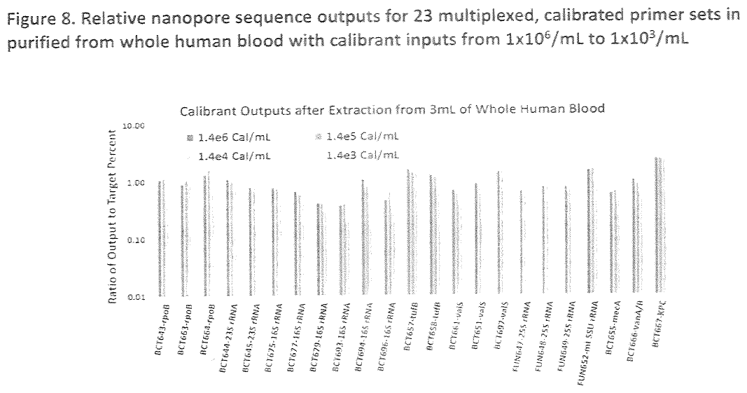

FIG. 8 shows the relative outputs of calibrant amplicons in the multiplexed PCR from varying calibrant mixture input levels into 3 mL of whole human blood.

FIG. 9 shows a device drawing for a 3-sample embodiment of the “MotoLyser” for disrupting difficult bacteria and fungi.

FIG. 10 shows photographs of the device produced through 3D printing and assembly of designed parts.

FIG. 11 shows a device controller interface written in C#.NET for operation in Windows OS through USB serial connection to an Arduino Nano or Elegoo Nano microcontroller on the assembled device.

FIG. 12 shows technical drawings for the parts to assemble the device depicted in FIG. 9 and FIG. 10. Distances shown are in millimeters and angles in degrees.

To assess the minimum number of sequences required to reproducibly achieve 100% sequence ID for a collection of nanopore sequences, nanopore sequences obtained for 10 regions amplified from MRSA extracted from a complex blood sample were analyzed. Sequence alignments automatically generated with the JIS analysis software were examined, and for each region 1000 bootstrapping trials were performed for each set of randomly-selected nanopore sequences ranging from one to 500 reads to examine the mean, median and mode of the percent ID and coverage of consensus sequences relative to the known S. aureus and mecA sequences.

Table 3 shows that, although individual sequences have considerable error, all regions except one require fewer than 15 nanopore reads to generate a consensus sequence that is 100% accurate a majority of the time. All regions except one generate a consensus sequence with 100% accuracy 100% of the time (out of 1000 random trials) with fewer than 500 sequences, and all regions will generate a 100% accurate consensus >95% of the time with fewer than 500 sequence reads (most of the time, 10 sequences will generate a consensus with 100% ID). Note that the assessment in Table 3 is performed only over the internal amplified sequence regions, excluding the primer-incorporated and library preparation-induced sequences, which also helps increase the overall sequence accuracy by excluding the proximal terminal regions that both have higher error rates and are uninformative about the amplified sequences.

A software package, NanoSepsID, has been developed by JIS for Windows operating systems that fully automates the operation and analysis of nanopore sequencing derived from our bloodborne pathogen identification assay. The software comprises 1.) a core logic module (NanoSepsIDCore) consisting of namespaces, classes, properties and methods to handle nanopore sequencing control, data analysis, configurations, databases, indexes, input, output and reporting functions, 2.) A GUI interface that allows the user to operate the software, conduct nanopore sequencing, interface with analysis parameters, display and browse analysis reports and perform utility functions such as monitoring the sequencing device and backing up data; and 3.) a command-line interface that allows headless analysis operation and scripting of reanalysis of samples (e.g., with modified parameters/database),

TABLE 1
Molecular targets of the JIS blood-borne infection assay version 1^{#}

| | Moecular | Target | Reference | Number of | |
|---|---|---|---|---|---|
| Category | Target | Organisms | Sequence | primers | Coordinates |
| Bacteria | 16S rRDA | All Bacteria | NC_008253.1 | 6 | 2739928 . . . 2740027 |
| | | | | | 2739928 . . . 2740292 |
| | | | | | 2739928 . . . 2740414 |
| | | | | | 2740176 . . . 2740292 |
| | | | | | 2740176 . . . 2740414 |
| | | | | | 2740306 . . . 2740414 |
| | 23S rDNA | | NR_103073.1 | 2 | 1825 . . . 1935 |
| | | | | 2 | 238 . . . 484 |
| | rpoB | Proteobacteria | NZ_UN849008.1 | 2 | 344828 . . . 345589 |
| | | | | 2 | 346730 . . . 346924 |
| | | | | 3 | 348236 . . . 348422 |
| | tufB | Firmicutes, | NC_021670.1 | 2 | 636315 . . . 637008 |
| | | Bacteroidetes, | | | |
| | | Proteobacteria | | | |
| | valS | Gamma-proteobateria | X05891.1 | 2 | 243 . . . 667 |
| Drug | KPC | N/A | AY034847.1 | 2 | 226 . . . 838 |
| Resistance | vanA/vanB | | M97297.1 | 2 | 7713 . . . 7953 |
| | mecA | | Y14051.1 | 2 | 3783 . . . 4109 |
| Yeast/ | 25S rDNA | Ascomycota, | | 2 | 134 . . . 261 |
| Fungi | | Mucormycota, | X70659.1 | 2 | 697 . . . 834 |
| | | Basidiomycota | | 2 | 2470 . . . 2617 |
| | Mitochondrial | Candida spp. | AF285261.1 | 2 | 27683 . . . 27979 |
| | SSU rDNA | | | | |

^{#}Reference sequence refers to accession number available in NCBI database with coordinates referring to location of the identifier nucleic acid sequences.

TABLE 2
Exemplary nucleic acid sequences of primers and primer pairs for detection
and identification of an unidentified microbial agent with [symbol] blood-borne infection
assay version 1*

| [symbol] | [symbol] | [symbol] |
|---|---|---|
| [symbol] | [symbol] | [symbol] |
| [symbol] | [symbol] | [symbol] |
| [symbol] | [symbol] | [symbol] |
| | | [symbol] |
| [symbol] | [symbol] | [symbol] |
| [symbol] | [symbol] | [symbol] |
| [symbol] | [symbol] | [symbol] |
| | [symbol] | [symbol] |
| | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

*/5Biosg/indicates biotin conjugated at the 5′ end. [symbol]
indicates data missing or illegible when filed

TABLE 3
Statistical assessment of nanopore sequencing accuracy
when analyzed in aggregate with consensus sequences

| | | | Lowest | Lowest | Lowest | Minimum number of | | |
|---|---|---|---|---|---|---|---|---|
| | | Mean % ID | % ID in | % ID in | % ID in | sequences to achieve | | |
| | Amplified | for 1000 | 1000 | 1000 | 1000 | | | Mean |
| Sequence | Region | individual | trials of | trials of | trials of | Mode of | Median of | with 0 |
| Region | Length | sequences | 5 reads | 10 reads | 100 reads | 100% ID | 100% ID | stddev |
| 16S rDNA 1 | 444 | 93.5 ± 1.9 | 96.62 | 98.2 | 99.55 | 30 | 35 | NR* |
| 16S rDNA 2 | 56 | 97.1 ± 2.9 | 94.64 | 98.21 | 100 | 3 | 3 | 17 |
| 16S rDNA 3 | 326 | 94.0 ± 1.9 | 96.32 | 98.16 | 99.39 | 13 | 15 | 120 |
| 16S rDNA 4 | 62 | 95.8 ± 2.9 | 95.16 | 96.77 | 100 | 3 | 3 | 20 |
| 16S rDNA 5 | 76 | 95.0 ± 2.7 | 96.05 | 97.37 | 100 | 5 | 5 | 70 |
| 16S rDNA 6 | 194 | 94.5 ± 2.2 | 96.39 | 97.94 | 99.48 | 7 | 8 | 460 |
| 23S rDNA 1 | 64 | 96.2 ± 2.7 | 96.88 | 98.44 | 100 | 3 | 3 | 13 |
| 23S rDNA 2 | 243 | 95.0 ± 2.7 | 97.12 | 98.77 | 100 | 5 | 7 | 40 |
| tufB | 649 | 93.2 ± 2.2 | 96.61 | 98.92 | 99.85 | 11 | 13 | 165 |
| mecA | 274 | 94.0 ± 2.6 | 96.72 | 98.18 | 99.64 | 11 | 9 | 360 |

*One out of ten regions did not display 1000/1000 consensus sequences with 100% ID to the known sequence. For the longest 16S rDNA product, the lowest % ID from 1000 random selections of 220 sequences was 99.8% and 95% of 1000 trials of randomly-selected nanopore reads generate a consensus sequence with 100% ID after 400 nanopore reads.

TABLE 4
Molecular targets of the JIS blood-borne infection assay version 2^{#}

| | Molecular | Target | Reference | Number of | |
|---|---|---|---|---|---|
| Category | Target | Organisms | Sequence | Primers | Coordinates |
| Bacteria | 16SrDNA | All Bacteria | NC_008253.1 | 6 | 2737826 . . . 2737934 |
| | | | | | 2737826 . . . 2738064 |
| | | | | | 2737934 . . . 2738064 |
| | | | | | 2737826 . . . 2738314 |
| | | | | | 2738213 . . . 2738314 |
| | | | | | 2737934 . . . 2738314 |
| | 23S rDNA | | NR_103073.1 | 2 | 1825 . . . 1935 |
| | | | | 2 | 238 . . . 484 |
| | rpoB | Proteobacteria | NZ_LN849008.1 | 3 | 348236 . . . 348422 |
| | | | NZ_CP007592.1 | 2 | 4761939 . . . 4762244 |
| | | | | 3 | 4763260 . . . 4763508 |
| | tufB | Firmicutes, | NC_21670.1 | 2 | 636087 . . . 636329 |
| | | Bacteroidetes, | | 2 | 636979 . . . 637188 |
| | valS | Gamma | X05891.1 | 2 | 956 . . . 1294 |
| | | Proteobacteria, | | 3 | 243 . . . 667 |
| | | Enterobacteriaceae | | | 243 . . . 373 |
| Drug Resistance | KPC | N/A | AY034847.1 | 2 | 121 . . . 309 |
| | vanA/vanB | | M97297.1 | 2 | 7715 . . . 7958 |
| | mecA | | Y14051.1 | 2 | 3783 . . . 4109 |
| Yeast/ | 25S rDNA | Ascomycota, | X70659.1 | 2 | 134 . . . 261 |
| Fungi | | Mucormycota, | | 2 | 697 . . . 834 |
| | | Basidiomycota | | 2 | 2470 . . . 2617 |
| | Mitochondrial | Candida spp. | AF285261.1 | 2 | 27683 . . . 27979 |
| | SSU rDNA | | | | |
| Process Control | Synthetic Construct | Process Control | N/A (synthetic) | 2 | 95 . . . 258 |

^{#}Reference sequence refers to accession number available in NCBI database with coordinates referring to location of the identifier nucleic acid sequences.

TABLE 5
Exemplary nucleic acid sequences of primers and primer pairs for detection
and identification of an unidentified microbial agent with [symbol] blood-borne infection
assay version 2*

| [symbol] | [symbol] | [symbol] |
|---|---|---|
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| | [symbol] | [symbol] |
| [symbol] | [symbol] | [symbol] |
| [symbol] | [symbol] | [symbol] |
| | | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |
| [symbol] | [symbol] | [symbol] |

*/5Biosg/indicates biotin conjugated at the 5′ end. [symbol]
indicates data missing or illegible when filed

TABLE 6
Exemplary nucleic acid sequences of synthetic calibrafors for quantification of
microbial agent with [symbol] blood-borne infection assay version 2.

| | [symbol] | [symbol] | | |
|---|---|---|---|---|
| [symbol] | [symbol] | [symbol] | [symbol] | [symbol] |
| [symbol] | [symbol] | [symbol] | [symbol] | [symbol] |
| | | | [symbol] | [symbol] |
| | | | [symbol] | [symbol] |
| | | | [symbol] | [symbol] |
| | | | [symbol] | [symbol] |
| | | | [symbol] | [symbol] |
| | | | | [symbol] |
| [symbol] | [symbol] | [symbol] | [symbol] | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| | | | | [symbol] |
| | | | | [symbol] |
| [symbol] | [symbol] | [symbol] | [symbol] | [symbol] |
| | | | [symbol] | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| | | | | [symbol] |
| [symbol] | [symbol] | [symbol] | [symbol] | [symbol] |
| | | | | [symbol] |
| | | | | [symbol] |
| [symbol] | [symbol] | [symbol] | [symbol] | [symbol] |
| | | | | [symbol] |
| | | | | [symbol] |
| [symbol] | [symbol] | [symbol] | [symbol] | [symbol] |
| | | | | [symbol] |
| | | | | [symbol] |
| [symbol] | [symbol] | [symbol] | [symbol] | [symbol] |
| | | | | [symbol] |
| | | | | [symbol] |
| | | | | [symbol] |
| | | | | [symbol] |

[symbol]

indicates data missing or illegible when filed

TABLE 7
By way of example only, and not limited to, selected Bacteria
and Fungi that may be identified with the invention

| Kingdom | Phylum | Family | Genus | Species |
|---|---|---|---|---|
| Bacteria | Actinobacteria | Acidimicrobiaceae | Acidimicrobium | Acidimicrobium |
| | | | | ferrooxidans |
| | | | Actinomyces | Actinomyces slackii |
| | | | | Actinomyces viscosus |
| | | Bifidobacteriaceae | Bifidobacterium | Bifidobacterium |
| | | | | adolescentis |
| | | | | Bifidobacterium |
| | | | | bifidum |
| | | | | Bifidobacterium |
| | | | | gallinarum |
| | | Corynebacteriaceae | Corynebacterium | Corynebacterium |
| | | | | diphtheriae |
| | | | | Corynebacterium |
| | | | | flavescens |
| | | | | Corynebacterium |
| | | | | glutamicum |
| | | | | Corynebacterium |
| | | | | pseudotuberculosis |
| | | | | Corynebacterium |
| | | | | simulans |
| | | | | Corynebacterium |
| | | | | ulcerans |
| | | Gordoniaceae | Gordonia | Gordonia bronchialis |
| | | Mycobacteriaceae | Mycobacterium | Mycobacterium avium |
| | | | | Mycobacterium |
| | | | | haemophilum |
| | | | | Mycobacterium leprae |
| | | | | Mycobacterium |
| | | | | tuberculosis |
| | | Nocardiaceae | Nocardia | Nocardia nova |
| | | | Rhodococcus | Rhodococcus fascians |
| | | Brevibacteriaceae | Brevibacterium | Brevibacterium |
| | | | | aurantiacum |
| | | Microbacteriaceae | Microbacterium | Microbacterium |
| | | | | hominis |
| | | Micrococcaceae | Rothia | Rothia dentocariosa |
| | Bacteroidetes | Bacteroidaceae | Bacteroides | Bacteroides caccae |
| | | | | Bacteroides fragilis |
| | | | | Bacteroides |
| | | | | thetaiotaomicron |
| | | N/A | Phocaeicola | Bacteroides vulgatus |
| | | Porphyromonadaceae | Porphyromanas | Porphyromonas |
| | | | | gingivalis |
| | | Prevotellaceae | Prevotella | Prevotella dentalis |
| | | | | Prevotella denticola |
| | | | | Prevotella intermedia |
| | | | | Prevotella jejuni |
| | | Blattabacteriaceae | Blattabacterium | Blattabacterium |
| | | | | clevelandi |
| | | Flavobacteriaceae | Capnocytophaga | Capnocytophaga |
| | | | | gingivalis |
| | | | Flavobacterium | Flavobacterium |
| | | | | branchiophilum |
| | | | | Flavobacterium |
| | | | | indicum |
| | | | | Flavobacterium |
| | | | | johnsoniae |
| | | | | Flavobacterium |
| | | | | pallidum |
| | Chlamydiae | Chlamydiaceae | Chlamydia | Chlamydia gallinocea |
| | | | | Chlamydia |
| | | | | pneumoniae |
| | | | | Chlamydia psittaci |
| | | | | Chlamydia trachomatis |
| | Firmicutes | Bacillaceae | Anoxybacillus | Anoxybacillus |
| | | | | flavithermus |
| | | | Bacillus | Bacillus anthracis |
| | | | | Bacillus licheniformis |
| | | | | Bacillus megaterium |
| | | | | Bacillus mycoides |
| | | | | Bacillus subtilis |
| | | | | Bacillus thuringiensis |
| | | | Metabacillus | Bacillus litoralis |
| | | Listeriaceae | Listeria | Listeria innocua |
| | | | | Listeria |
| | | | | monocytogenes |
| | | Paenibacillaceae | Paenibacillus | Paenibacillus lentus |
| | | Staphylococcaceae | Staphylococcus | Staphylococcus |
| | | | | argenteus |
| | | | | Staphylococcus aureus |
| | | | | Staphylococcus |
| | | | | auricularis |
| | | | | Staphylococcus capitis |
| | | | | Staphylococcus caprae |
| | | | | Staphylococcus |
| | | | | epidermidis |
| | | | | Staphylococcus |
| | | | | haemolyticus |
| | | | | Staphylococcus |
| | | | | hominis |
| | | | | Staphylococcus kloosii |
| | | | | Staphylococcus |
| | | | | lugdunensis |
| | | | | Staphylococcus lutrae |
| | | | | Staphylococcus |
| | | | | schleiferi |
| | | | | Staphylococcus sciuri |
| | | | | Staphylococcus |
| | | | | succinus |
| | | | | Staphylococcus |
| | | | | warneri |
| | | Aerococcaceae | Aerococcus | Aerococcus urinae |
| | | Enterococcaceae | Enterococcus | Enterococcus faecalis |
| | | | | Enterococcus faecium |
| | | | | Enterococcus |
| | | | | gallinarum |
| | | Lactobacillaceae | Lacticaseibacillus | Lactobacillus |
| | | | | rhamnosus |
| | | | Lactobacillus | Lactobacillus |
| | | | | acidophilus |
| | | | | Lactobacillus |
| | | | | delbrueckii |
| | | | | Lactobacillus johnsonii |
| | | | Lentilactobacillus | Lactobacillus buchneri |
| | | | Loigolactobacillus | Lactobacillus backii |
| | | Leuconostocaceae | Leuconostoc | Leuconostoc kimchii |
| | | Streptococcaceae | Lactococcus | Lactococcus lactis |
| | | | Streptococcus | Streptococcus |
| | | | | agalactiae |
| | | | | Streptococcus australis |
| | | | | Streptococcus |
| | | | | dysgalactiae |
| | | | | Streptococcus gordonii |
| | | | | Streptococcus |
| | | | | lutetiensis |
| | | | | Streptococcus mitis |
| | | | | Streptococcus mutans |
| | | | | Streptococcus oralis |
| | | | | Streptococcus |
| | | | | posteurianus |
| | | | | Streptococcus |
| | | | | pneumoniae |
| | | | | Streptococcus |
| | | | | pyogenes |
| | | | | Streptococcus |
| | | | | salivarius |
| | | | | Streptococcus |
| | | | | sanguinis |
| | | | | Streptococcus urinalis |
| | | | | Streptococcus viridans |
| | | Clostridiaceae | Clostridium | Clostridium |
| | | | | acetobutylicum |
| | | | | Clostridium botulinum |
| | | | | Clostridium |
| | | | | perfringens |
| | | | | Clostridium |
| | | | | sporogenes |
| | | Peptococcaceae | Desulfitobacterium | Desulfitobacterium |
| | | | | hafniense |
| | | Peptostreptococcaceae | Acetoanaerobium | Acetoanaerobium |
| | | | | sticklandii |
| | | Peptostreptococcaceae | Clostridioides | Clostridioides difficile |
| | Fusobacteria | Fusobacteriaceae | Fusobacterium | Fusobacterium |
| | | | | nucleatum |
| | | | | Fusobacterium |
| | | | | ulcerans |
| | Proteobacteria | Caulobacteraceae | Caulobacter | Caulobacter mirabilis |
| | | Bartonellaceae | Bartonella | Bartonella henselae |
| | | | | Bartonella quintana |
| | | | | Bartonella vinsonii |
| | | Acetobacteraceae | Acidiphilium | Acidiphilium |
| | | | | multivorum |
| | | Anaplasmataceae | Anaplasma | Anaplasma ovis |
| | | | Ehrlichia | Ehrlichia chaffeensis |
| | | Rickettsiaceae | Rickettsia | Rickettsia japonica |
| | | | | Rickettsia prowazekii |
| | | | | Rickettsia rickettsii |
| | | Sphingomonadaceae | Sphingomonas | Sphingomonas sp. |
| | | | | AAP5 |
| | | | Zymomonas | Zymomonas mobilis |
| | | Alcaligenaceae | Achromobacter | Achromobacter |
| | | | | denitrificans |
| | | | Bordetella | Bordetella bronchialis |
| | | | | Bordetella |
| | | | | bronchiseptica |
| | | | | Bordetella |
| | | | | parapertussis |
| | | | | Bordetella pertussis |
| | | Burkholderiaceae | Burkholderia | Burkholderia ambifaria |
| | | | | Burkholderia |
| | | | | contaminans |
| | | | | Burkholderia mallei |
| | | | | Burkholderia |
| | | | | pseudomallei |
| | | | | Burkholderia |
| | | | | thailandensis |
| | | | Paraburkholderia | Paraburkholderia |
| | | | | fungorum |
| | | | Ralstonia | Ralstonia |
| | | | | solanacearum |
| | | Comamonadaceae | Acidovorax | Acidovorax citrulli |
| | | Neisseriaceae | Neisseria | Neisseria elongata |
| | | | | Neisseria gonorrhoeae |
| | | | | Neisseria meningitidis |
| | | Azonexaceae | Dechloromonas | Dechloromonas |
| | | | | aromatica |
| | | Campylobacteraceae | Campylobacter | Campylobacter coli |
| | | | | Campylobacter jejuni |
| | | | | Campylobacter lari |
| | | | | Campylobacter |
| | | | | ureolyticus |
| | | Helicobacteraceae | Helicobacter | Helicobacter pylori |
| | | Aeromonadaceae | Aeromonas | Aeromonas |
| | | | | salmonicida |
| | | | | Aeromonos veronii |
| | | | Zobellella | Zobellella denitrificans |
| | | Pseudoalteromonadaceae | Pseudoalteromonas | Pseudoalteromonas |
| | | | | marina |
| | | Shewanellaceae | Shewanella | Shewanella |
| | | | | denitrificans |
| | | | | Shewanella |
| | | | | halifaxensis |
| | | Enterobacteriaceae | Citrobacter | Citrobacter braakii |
| | | | | Citrobacter freundii |
| | | | | Citrobacter pasteurii |
| | | | | Citrobacter youngae |
| | | | Enterobacter | Enterobacter |
| | | | | bugandensis |
| | | | | Enterobacter cloacae |
| | | | | Enterobacter ludwigii |
| | | | Escherichia | Escherichia coli |
| | | | | Escherichia fergusonii |
| | | | Klebsiella | Klebsiella oxytoca |
| | | | | Klebsiella pneumoniae |
| | | | | Klebsiella variicola |
| | | | Kluyvera | Kluyvera intermedia |
| | | | Salmonella | Salmonella bongori |
| | | | | Salmonella enterica |
| | | | Shigella | Shigella boydii |
| | | | | Shigella dysenteriae |
| | | | | Shigella flexneri |
| | | | | Shigella sonnei |
| | | Erwiniaceae | Erwinia | Erwinia persicina |
| | | Hafniaceae | Edwardsiella | Edwardsiella tarda |
| | | Morganellaceae | Proteus | Proteus mirabilis |
| | | | | Proteus vulgaris |
| | | Versiniaceae | Serratia | Serratia liquefaciens |
| | | | | Serratia marcescens |
| | | | | Serratia quinivorans |
| | | | | Yersinia enterocolitica |
| | | | | Yersinia pestis |
| | | | | Yersinia |
| | | | | pseudotuberculosis |
| | | | | Yersinia ruckeri |
| | | Coxiellaceae | Coxiella | Coxiella burnetii |
| | | Legionellaceae | Legionella | Legionella israelensis |
| | | | | Legionella |
| | | | | pneumophila |
| | | Pasteurellaceae | Actinobacillus | Actinobacillus |
| | | | | pleuropneumoniae |
| | | | | Actinobacillus |
| | | | | succinogenes |
| | | | Haemophilus | Haemophilus |
| | | | | influenzae |
| | | | | Haemophilus |
| | | | | parainfluenzae |
| | | | Pasteurella | Pasteurella mulocida |
| | | Moraxellaceae | Acinetobacter | Acinetobacter |
| | | | | baumannii |
| | | | | Acinetobacter |
| | | | | calcoaceticus |
| | | | | Acinetobacter |
| | | | | guillouiae |
| | | | | Acinetobacter |
| | | | | haemolyticus |
| | | | | Acinetobacter lactucae |
| | | | | Acinetobacter |
| | | | | schindleri |
| | | | Moraxella | Moraxella catarrhalis |
| | | | | Moraxella cuniculi |
| | | Pseudomonadaceae | Pseudomonas | Pseudomonas |
| | | | | aeruginosa |
| | | | | Pseudomonas |
| | | | | fluorescens |
| | | | | Pseudomonas |
| | | | | parafulva |
| | | | | Pseudomonas putida |
| | | | | Pseudomonas stutzeri |
| | | Francisellaceae | Francisella | Francisella noatunensis |
| | | | | Francisella tularensis |
| | | Vibrionaceae | Aliivibrio | Vibrio fischeri |
| | | | Vibrio | Vibrio cholerae |
| | | | | Vibrio |
| | | | | parahaemolyticus |
| | | | | Vibrio vulnificus |
| | | Xanthomonadaceae | Xanthomonas | Xanthomonas |
| | | | | campestris |
| | | | | Xanthomonas |
| | | | | perforans |
| | Spirochaetes | Brachyspiraceae | Brachyspira | Brachyspira murdochii |
| | | Leptospiraceae | Leptospira | Leptospira interrogans |
| | | | | Leptospira santarosai |
| | | Borreliaceae | Borrelia | Borrelia hermsii |
| | | | Borreliella | Borreliella burgdorferi |
| | | Spirochaetaceae | Treponema | Treponema caldarium |
| | | | | Treponema pallidum |
| | Tenericutes | Acholeplasmataceae | Acholeplasma | Acholeplasma oculi |
| | | Spiroplasmataceae | Spiroplasma | Spiroplasma citri |
| | | | | Spiroplasma |
| | | | | gladiatoris |
| | | | | Spiroplasma kunkelii |
| | | Mycoplasmataceae | Mycoplasma | Mycoplasma hominis |
| | | | | Mycoplasma ovis |
| | | | | Mycoplasma parvum |
| | | | | Mycoplasma |
| | | | | pneumoniae |
| | | | | Ureaplasma |
| | | | | urealyticum |
| Fungi | Ascomycota | Debaryomycetaceae | Candida | Candida parapsilosis |
| | | | | Candida orthopsilosis |
| | | | | Candida metapsilosis |
| | | Aspergillaceae | Aspergillus | Aspergillus clavatus |
| | | | | Aspergillus fumigatus |
| | | | | Aspergillus niger |
| | | | | Aspergillus oryzae |
| | | | | Aspergillus terreus |
| | | | Penicillium | Penicillium |
| | | | | chrysogenum |
| | | | | Penicillium verrucosum |
| | | Ajellomycetaceae | Histoplasma | Histoplasma |
| | | | | capsulatum |
| | | Pneumocystidaceae | Pneumocystis | Pneumocystis carinii |
| | | | | Pneumocystis jirovecii |
| | | Debaryomycetaceae | Candida | Candida albicans |
| | | | | Candida dubliniensis |
| | | | | Candida tropicalis |
| | | | Hyphopichia | Candida rhagii |
| | | | Teunomyces | Candida sake |
| | | Metschnikowiaceae | Clavispora | Candida auris |
| | | | | Candida blattae |
| | | | | Candida intermedia |
| | | Saccharomycetaceae | Kluyveromyces | Kluyveromyces lactis |
| | | | | Kluyveromyces |
| | | | | nonfermentans |
| | | | Kluyveromyces | Candida castellii |
| | | | | Candida glabrata |
| | | | Saccharomyces | Saccharomyces |
| | | | | cerevisiae |
| | | Schizosaccharomycetaceae | Schizosaccharomyces | Schizosaccharomyces |
| | | | | japonicus |
| | | Nectriaceae | Fusarium | Fusarium oxysporum |
| | | | | Fusarium secorum |
| | | | | Fusarium verrucosum |
| | | Microascaceae | Scedosporium | Scedosporium boydii |
| | Basidiomycota | Malasseziaceae | Malassezia | Malassezia dermatis |
| | | | | Malassezia furfur |
| | | | | Malassezia |
| | | | | pachydermatis |
| | Mucoromycota | Cunninghamellaceae | Cunninghamella | Cunninghamella |
| | | | | bertholletiae |
| | | Mucoraceae | Mucor | Mucor racemosus |

### REFERENCES

1. Principles and procedures for Blood Cultures; Approved Guideline, CLSI document M47-A. Wayne, P A: Clinical and Laboratory Standards Institute (CLSI); 2007.
2. Abril M K, Barnett A S, Wegermann K., et al. Diagnosis of Capnocytophaga canimorsus Sepsis by Whole-Genome Next-Generation Sequencing. Open Forum Infect Dis. 2016:3(3):ofw144.
3. Agyepong N, Govinden U, Owusu-Ofori A, Essack S Y. Multidrug-resistant gram-negative bacterial infections in a teaching hospital in Ghana. Antimicrob Resist Infect Control. 2018; 7:37.
4. Asif A A, Roy M, Ahmad S. Rare case of Ureaplasma parvum septic arthritis in an immunocompetent patient. BMJ Case Rep. 2020; 13(9).
5. Carrara L, Navarro F, Turbau M, et al. Molecular diagnosis of bloodstream infections with a new dual-priming oligonucleotide-based multiplex PCR assay. J Med Microbiol. 2013;62(Pt 11):1673-1679.
6. Cockerill F R, 3rd, Wilson J W, Vetter E A, et al. Optimal testing parameters for blood cultures. Clin Infect Dis. 2004; 38(12): 1724-1730.
7. de Alegria Puig C R, Torres M F, Marfil-Perez E, et al. Comparison between Vitek M S, Bruker Biotyper, Vitek2, and API20E for differentiation of species of the genus Raoultella. Eur J Clin Microbiol Infect Dis. 2019:38(3):467-470.
8. De Angelis G, Posteraro B, De Carolis E, et al. T2Bacteria magnetic resonance assay for the rapid detection of ESKAPEe pathogens directly in whole blood. J Antimicrob Chemother. 2018;73(suppl_4):iv20-iv26.
9. Denina M, Scolfaro C, Colombo S, et al. Magicplex™ Sepsis Real-Time test to improve bloodstream infection diagnostics in children. Eur J Pediatr. 2016; 175(8):1107-1111.
10. Ecker D J, Sampath R. Massire C, et al. Ibis T5000: a universal biosensor approach for microbiology. Nat Rev Microbiol. 2008;6(7):$53-558.
11. Evans S R. Hojer A M. Jiang H, et al. Informing Antibiotic Treatment Decisions: Evaluating Rapid Molecular Diagnostics To Identify Susceptibility and Resistance to Carbapenems against Acinetobacter spp. in PRIMERS III. J Clin Microbiol. 2017; 55(1):134-144.
12. Evans S R. Hujer A M, Jiang H, et al. Rapid Molecular Diagnostics, Antibiotic Treatment Decisions, and Developing Approaches to Inform Empiric Therapy: PRIMERS I and I1. Clin Infect Dis. 2016:62(2): 181-189.
13. Evans S R. Tran T T T, Hujer A M, et al. Rapid Molecular Diagnostics to Inform Empiric Use of Ceftazidime/Avibactam and Ceftolozane/Tazobactam Against Pseudomonas aeruginosa: PRIMERS IV. Clin Infect Dis. 2019; 68(11): 1823-1830.
14. Giannella M. Paolucci M, Roncarati G, et al. Potential role of T2Candida in the management of empirical antifungal treatment in patients at high risk of candidaemia: a pilot single-centre study. J Antimicrob Chemother. 2018; 73(10):2856-2859.
15. Goggin K P, Gonzalez-Pena V. Inaba Y. et al. Evaluation of Plasma Microbial Cell-Free DNA Sequencing to Predict Bloodstream Infection in Pediatric Patients With Relapsed or Refractory Cancer. JAMA Oncol. 2020; 6(4):552-556.
16. Ha J. Hong S K, Han G H. Kim M, Yong D, Lee K. Same-Day Identification and Antimicrobial Susceptibility Testing of Bacteria in Positive Blood Culture Broths Using Short-Term Incubation on Solid Medium with the MicroFlex L T, Vitek-MS, and Vitek2 Systems. Ann Lab Med. 2018:38(3):235-241.
17. Heireman L, Patteet S. Steyaert S. Performance of the new ID-fungi plate using two types of reference libraries (Bruker and MSI) to identify fungi with the Bruker MALDI Biotyper. Med Mycol. 2020; 58(7):946-957.
18. Hu Y L, Hsueh S C, Ding G S, et al. Applicability of an in-house saponin-based extraction method in Bruker Biotyper matrix-assisted laser desorption/ionization time-of-flight mass spectrometry system for identifying bacterial and fungal species in positively flagged pediatric VersaTREK blood cultures, J Microbiol Immunol Infect. 2020:53(6):916-924.
19. Jacob D, Sauer U, Housley R, et al. Rapid and high-throughput detection of highly pathogenic bacteria by Ibis PLEX-ID technology. PLOS One. 2012; 7(6):e39928.
20 Jordana-Lluch E. Gimenez M, Quesada M D. et al. Evaluation of the Broad-Range PCR/ESI-MS Technology in Blood Specimens for the Molecular Diagnosis of Bloodstream Infections. PLOS One. 2015;10(10):e0140865.
21. Kaleta E J, Clark A E, Cherkaoui A, et al. Comparative analysis of PCR-electrospray ionization/mass spectrometry (MS) and MALDI-TOF/MS for the identification of bacteria and yeast from positive blood culture bottles. Clin Chem. 2011; 57(7): 1057-1067.
22. Liong M, Hoang A N, Chung J, et al. Magnetic barcode assay for genetic detection of pathogens. Nat Commun. 2013:4:1752.
23. Loonen A J, de Jager C P, Tosserams J, et al. Biomarkers and molecular analysis to improve bloodstream infection diagnostics in an emergency care unit. PLOS One. 2014;9(1):e87315.
24. Marco F. Molecular methods for septicemia diagnosis. Enferm Infece Microbiol Clin. 2017:35(9):586-592.
25. Mermel L A, Maki D G, Detection of bacteremia in adults: consequences of culturing an inadequate volume of blood. Ann Intern Med. 1993; 119(4):270-272.
26. Metzgar D, Frinder M W, Rothman R E, et al. The IRIDICA BAC BSI Assay: Rapid. Sensitive and Culture-Independent Identification of Bacteria and Candida in Blood. PLOS One. 2016;11(7)e0158186.
27. Munoz P, Vena A, Machado M, et al. T2Candida MR as a predictor of outcome in patients with suspected invasive candidiasis starting empirical antifungal treatment: a prospective pilot study. J Antimicrob Chemother. 2018;73(suppl_4):iv6-iv12.
28. Munoz P, Vena A, Machado M, et al. T2MR contributes to the very early diagnosis of complicated candidaemia. A prospective study. J Antimicrob Chemother 2018;73(suppl_4):iv13-iv19.
29. Mylonakis E, Clancy C J, Ostrosky-Zeichner L, et al. T2 magnetic resonance assay for the rapid diagnosis of candidemia in whole blood: a clinical trial. Clin Infect Dis. 2015; 60(6):892-899.
30. Neely L A, Audeh M, Phung N A, et al. T2 magnetic resonance enables nanoparticle . . . mediated rapid detection of candidemia in whole blood. Sci Transl Med. 2013;5(182): 182ra154.
31. Nguyen M H, Clancy C J, Pasculle A W, et al. Performance of the T2Bacteria Panel for Diagnosing Bloodstream Infections: A Diagnostic Accuracy Study. Ann Intern Med. 2019; 170(12):845-852.
32. Ozenci V, Patel R, Ullberg M, Stralin K. Demise of Polymerase Chain Reaction/Electrospray Ionization-Mass Spectrometry as an Infectious Diseases Diagnostic Tool. Clin Infect Dis. 2018; 66(3):452-4SS.
33. Saffert R T, Cunningham S A, Ihde S M, Jobe K E, Mandrekar J, Patel R. Comparison of Bruker Biotyper matrix-assisted laser desorption ionization-time of flight mass spectrometer to BD Phoenix automated microbiology system for identification of gram-negative bacilli. J Clin Microbiol. 2011; 49(3):887-892.
34, Stevenson M, Pandor A, Martyn-St James M, et al. Sepsis: the LightCycler SeptiFast Test MGRADE®, SepsiTest and IRIDICA BAC BSI assay for rapidly identifying bloodstream bacteria and fungi—a systematic review and economic evaluation. Health Technol Assess. 2016:20(46): 1-246.
35 Tassinari M, Zannoli S. Farabegoli P. et al. Rapid diagnosis of bloodstream infections in the critically ill: Evaluation of the broad range PCR/ESI-MS technology. PLOS One. 2018:13(5):e0197436.
36. Voigt C. Silbert S, Widen R H, et al. The T2Bacteria Assay Is a Sensitive and Rapid Detector of Bacteremia That Can Be Initiated in the Emergency Department and Has Potential to Favorably Influence Subsequent Therapy. J Emerg Med. 2020; 58(S): 785-796.
37. Wang H, Li Y. Fan X, Chiueh T S, Xu Y C, Hsueh P R. Evaluation of Bruker Biotyper and Vitek MS for the identification of Candida tropicalis on different solid culture media. J Microbiol Immunol Infect. 2019; 52(4):604-611.
38. Wiesinger-Mayr H, Vierlinger K, Pichler R, et al. Identification of human pathogens isolated from blood using microarray hybridisation and signal pattern recognition. BMC Microbiol. 2007;7:78.
39. Zboromyrska Y, Cilloniz C. Cobos-Trigueros N, et al. Evaluation of the Magicplex Sepsis Real-Time Test for the Rapid Diagnosis of Bloodstream Infections in Adults. Front Cell Infect Microbiol. 2019; 9:56.

1. A method for detecting the presence, identifying and/or quantifying an amount of an unidentified microbial agent in a sample comprising:
a) Mixing a sample with quantified calibrator molecules;
b) Isolating nucleic acid in the sample;
c) Interrogating and amplifying select nucleic acid sequences with a collection of primer pairs so as to produce double-stranded DNAs, wherein each double-stranded DNA comprises an identifier nucleic acid sequence, a universal amplification target sequence, and a chemical tag;
d) Isolating the double-stranded DNAs so produced in step (c) using the chemical tag;
e) Determining DNA sequence of the isolated double-stranded DNAs;
f) Identifying and quantifying the output of the calibrator molecules added in step (a);
g) Organizing DNA sequences so obtained in step (e) to produce a collection of consensus sequences;
h) Comparing the collection of consensus sequences against reference sequences so as to detect the presence of an unidentified microbial agent and identify the microbial agent in the sample; and
i) Optionally, comparing the signal output levels from each agent identified in the sample to the output levels of the calibrator molecules in order to quantify the genome copy amount of the agent in the original sample.

2. The method of claim 1, wherein the sample is in liquid.

3. The method of claim 1, wherein the sample is a liquid sample.

4. The method of claim 1, wherein the sample is a sample from a subject.

5. The method of claim 4, wherein the sample is blood.

6. The method of claim 4, wherein the subject is an animal.

7. The method of claim 6, wherein the animal is selected from the group consisting of bird, reptile, fish and mammal.

8. The method of claim 7, wherein the mammal is selected from the group consisting of mouse, rat, dog, cat, civet, pangolin, bat, pig, guinea pig, goat, sheep, donkey, horse, camel, chimpanzee, monkey, gorilla, cattle, and human.

9. The method of claim 8, wherein the mammal is human.

10. The method of claim 1, wherein isolating nucleic acid in the sample of step (b) comprises a pH-dependent nucleic acid binding to and release from a solid support.

11. The method of claim 10, wherein the nucleic acid binds to the solid support at a lower pH and detaches from the solid support at a higher pH.

12. The method of claim 1, wherein the nucleic acid is DNA.

13. The method of claim 10, further comprising the step of separating the unidentified microbial agent from the sample prior to step (b).

14. The method of claim 13, wherein the sample is a blood sample.

15. The method of claim 14, wherein the blood sample is a whole blood sample comprising blood cells.

16. The method of claim 15, wherein the blood cells are red blood cells.

17. The method of claim 16, wherein separating the unidentified microbial agent comprises contacting the blood sample with a starch so that red blood cells shrink in volume and aggregate clearing the blood sample by gravity sedimentation, thereby separating the unidentified microbial agent from the sample.

18. The method of claim 17, wherein the starch is hetastarch or hydroxy-ethyl starch (HES).

19. The method of claim 1, further comprising disrupting the microbial agent so that the agent fractures, exposes and releases its nucleic acid after step (a) and prior to step (b).

20. The method of claim 19, wherein disrupting involves the step of physically breaking up the unidentified microbial agent in an agitator.

21-51. (canceled)

52. (canceled)

53.-55. (canceled)

56.-68. (canceled)

69. (canceled)

70-75. (canceled)

76.-246. (canceled)
